(19) United States
(12) Patent Application Publication
(10) Pub. No.: US 2025/0164468 A1
(43) Pub. Date: May 22, 2025

(54) Method for Cellular Lifespan Measurement

(71) Applicant: AGENCY FOR SCIENCE, TECHNOLOGY AND RESEARCH, Singapore (SG)

(72) Inventors: Mohammad Alfatah, Singapore (SG); Frank Eisenhaber, Singapore (SG)

(21) Appl. No.: 18/839,124

(22) PCT Filed: Feb. 17, 2023
(86) PCT No.: PCT/SG2023/050097

§ 371 Date: Aug. 16, 2024

(30) Foreign Application Priority Data
Feb. 17, 2022 (SG) 10202201534P

(51) Int. Cl. G01N 33/50; C12Q 1/02; G01N 1/30

(52) U.S. Cl. CPC G01N 33/5091; C12Q 1/025; G01N 1/30; G01N 2001/302

(57) ABSTRACT

The present invention refers to a method for high-throughput screening of viability of cells, the method comprising the steps of: a) treating, in a well of a first multi-well plate, a sample containing cells with a condition that modulates lifespan; b) transferring a portion of the cells in the well of the first multi-well plate into a well of a second multi-well plate; c) mixing, in a well of the second multi-well plate, the portion of cells with a fluorescent viability dye to stain the cells; d) measuring fluorescence intensity of the stained cells in a plate reader to obtain a fluorescence intensity value; e) measuring optical density of the cells in the plate reader to obtain an optical density value; and f) normalizing the fluorescence intensity value with the optical density value, wherein the normalized fluorescence intensity value directly correlates with the viability of the cells.

### TECHNICAL FIELD

The present invention relates to a method for high-throughput screening to determine cellular lifespan.

### BACKGROUND ART

Besides generally reducing life activity, aging remains the greatest risk factor for the development of chronic diseases that, subsequently, compromise independent human life and finally lead to death. Since 1950, life expectancy has drastically increased from 47 years to 73 years. Despite the recent trend toward dwindling birth rates, the world population spiked from 2.9 billion in 1950 to 7.8 billion in 2020. This remarkable increase in human longevity results in a higher proportion of the population above 65 years of age, with almost one out of five in the most advanced countries. However, the increase in the number of years lived is not a representation of the health status of the elderly. There is a remarkable gap of almost ten years between the healthspan, which is the period essentially free from diseases, and the actual lifespan. Elders tend to have costly chronic diseases such as cancer, neurodegenerative and cardiovascular diseases and other metabolic syndromes, which severely hamper their quality of life and ability to work. As such, this puts tremendous pressure on the healthcare system and the working population. The current medical approaches to prevent age-related pathologies are recommendations for a healthy lifestyle, including exercise and diet. However, these interventions alone are not sufficient to prevent the onset of age-related diseases.

Aging is characterized by a progressive loss of physiological integrity, efficiency in cellular functions, and metabolic signalling. Increasing efforts are directed to understand cellular aging processes affecting the highly interconnected and functionally redundant gene and protein interactions network. Despite the complexity of aging, recent research in different model systems, including mammals, has demonstrated that delayed aging and increased healthspan are feasible by anti-aging interventions such as rapamycin drug administration and calorie (glucose) restriction.

Recent research has shown that molecular mechanisms observed in human aging are conserved in different organisms, including single-cell yeast. The budding yeast Saccharomyces cerevisiae is one of the most studied model organisms for uncovering the biological processes involved in cellular aging. The benefits of studying aging in yeast include a short generation time, a tractable lifespan, and its amenability to high-throughput assays. Therefore, this organism became a powerful tool for the identification of anti-aging interventions. Yeast is commonly used to study aging in two distinct ways: replicative lifespan (RLS) and chronological lifespan (CLS). The RLS measures the number of times an individual cell divides, an aging model for mitotic cells such as stem cells. The CLS measures the length of time a non-dividing cell remains viable in the stationary phase, an aging model for post-mitotic cells such as neurons and muscle cells. The viability of aging yeast cells under nutrient-deprived stationary phase conditions decreases, and they eventually die.

CLS has been traditionally measured by estimating colony-forming units (CFUs) counts on the agar plate in an outgrowth assay. Yeast cells in a flask with total media (≥20 mL) are exposed to a single chemical compound. At different time points (e.g. after 2, 5, and 8 days), small aliquots of the chronological aging culture from the flask are serially diluted, plated onto nutrient agar plates, and incubated for 2 to 3 days for colonies formation and counting. The survival fraction is determined from the CFU for each chronological age-point relative to day 1 (considered 100% cell survival). The CFU approach is quantitative but associated with many downsides. It requires multiple serial dilutions, plating steps, and incubation periods for the colonies to appear. It is dependent on the ability of the post-mitotic cells to re-enter into the mitotic phase. Colony counting is performed either by manual or expensive instruments. Moreover, this method is unsuitable and too expensive for high-throughput screening (HTS) of thousands of compounds because it requires substantial amounts of the drugs tested, large volume flask cultures, and many agar plates. Unfortunately, the CFU method and all recent variants for measuring CLS are critically dependent on the ability of the post-mitotic cells to re-enter into the mitotic phase. In principle, this methodology cannot distinguish yeast in mitotic arrest from dead cells.

Currently available CFU-derived methods measure cell survival and growth based on the outgrowth of drug-exposed stationary phase yeast in nutrient-rich culture either in liquid medium or by a spotting assay on an agar plate. After 24 hours, the outgrowth of aged cells in liquid medium is measured by the absorbance at OD600 nm, whereas in the spotting assay, the outgrowth of spotted aged culture onto agar medium is visually identified after 48 hours. Furthermore, for pooled tagged yeast deletion strains, flow cytometry may be necessary to quantify the individual strain viability.

Conventionally, CLS of human cells is measured by entering the human cells in a post-mitotic stage by nutrient deprivation under selected anti-aging experimental conditions. Survival of post-mitotic cells is then measured by outgrowth in a fresh medium. That is, exhausted medium (together with floating dead cells) is aspirated. Attached cells are trypsinized and transferred to 6-well plates with fresh medium. They are incubated for seven days before determining the cell viability via counting the number of grown colonies using crystal violet staining. This method has previously been applied to prove the anti-aging effect of rapamycin as well as the flavonoid 4,4′-dimethoxychalcone to promote CLS in cells of several species including human.

However, this method requires a long experiment time. At the outgrowth stage, the 6-well plates need seven days to reach sufficient cell count to be detected by the crystal violet assay. If primary fibroblasts were used instead of immortal cell lines, it would require an even longer growing period. Further, the method is not suitable for cells with a low adherent ability to the culture plate. The rinsing of cells before trypsinization and crystal violet staining would result in varying levels of cell loss due to detachment, leading to variations in the results. This method is also not suitable for high-throughput screening as the number of compounds and concentrations that can be tested are restricted by the plate capacity. The crystal violet assay produces image data for qualitative visual comparison. Thus, the qualitative nature of the data fails to capture minor changes in cell lifespan induced by attempted anti-aging interventions.

In view of the above, there is a need to develop a new quantification method to determine the lifespan of the aged cell that overcomes or at least ameliorates, one or more of the limitations described above.

### SUMMARY

In an aspect, there is provided a method for high-throughput screening to determine cellular lifespan, the method comprising the steps of:
a) treating, in a well of a first multi-well plate, a sample containing cells with a condition that modulates lifespan of the cells;
b) transferring a portion of the cells in the well of the first multi-well plate into a well of a second multi-well plate;
c) mixing, in a well of the second multi-well plate, the portion of cells with a fluorescent viability dye to stain the cells;
d) measuring fluorescence intensity of the stained cells in a plate reader to obtain a fluorescence intensity value;
e) measuring optical density of the cells in the plate reader to obtain an optical density value; and
f) normalizing the fluorescence intensity value with the optical density value, wherein the normalized fluorescence intensity value directly correlates with the viability of the cells.

In another aspect, there is provided a method for high-throughput screening to determine cellular lifespan, the method comprising the steps of:
a) plating, in a well of the multi-well plate, a sample containing cells and a fluorescent viability dye to stain the cells,
b) treating, in the well of the multi-well plate, the sample containing cells with a condition that modulates the lifespan of the cells; and
c) measuring fluorescence intensity of the stained cells in a plate reader to obtain a fluorescence intensity value.

Advantageously, the method may provide a cost- and time-efficient way to quantitatively screen the viability and chronological lifespan of cells. The method may be used in any kind of screening of cell viability following treatment with conditions that modulate the lifespan of cells to directly quantify cell survival. Advantageously, the method may not require an outgrowth assay. More advantageously, the method as defined above may not require additional steps of aspiration, washing, trypsinization of transferring of cells to multiple new plates. Therefore, the method may provide an alternative method for screening the lifespan of cells to the methods that are conventionally available, thereby circumventing excessive handling, time-consuming procedures and resource-intensive steps.

Advantageously, the method may only require equipment and reagents that are cheap and readily available. Moreover, the method may facilitate high-throughput screening by using multi-well plates, whereby the output of fluorescence and optical density may be determined immediately one after another within a matter of seconds or minutes, using a plate reader. Further advantageously, by eliminating a conventional out-growth step, the method may be a high-throughput method, which may enable the testing of cell viability in numerous samples at the same time, significantly reducing assay time and use of resources. Further advantageously, the entire method from step a) to e) may be performed within a matter of minutes.

Advantageously, the method may be a plate-based method to determine the chronological lifespan of cells based on (i) the ratio of dead/surviving cells and (ii) the total amount of cells, which may be derived simultaneously from the same plate using a microplate reader using fluorescence and optical density measurements. Further advantageously, by treating the cells in a first multi-well plate and transferring them to a second multi-well plate for the screening, it may allow screening of different lifespan-modulating conditions at different time points from the same sample. This may facilitate consistent screening from the same sample and minimize the errors involved in having to compare viability of cells treated with the lifespan modulating conditions in different plates.

More advantageously, the method as defined above may be performed independently of the ability of the cells to proliferate. In addition, the method as defined above may advantageously be performed on a variety of cell types, including fungal, bacterial and animal cells, regardless of whether they are primary cells or immortalised cells.

### Definitions

The following words and terms used herein shall have the meaning indicated:

The phrase “high-throughput screening”, in the context of the present disclosure, refers to the use of automated equipment to rapidly test the specific biological activity of chemical and/or biological compounds. High-throughput screening allows for the rapid testing of thousands to millions of chemical and/or biological compounds within a short time frame.

The phrase “chronological lifespan” in the context of the present disclosure, refers to the period of time that non-dividing cells remain alive in stationary phase or in nutrient deprived conditions.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

The abbreviation CLS stands for chronological lifespan, which is defined as the number of days cells remain viable in a stationary phase.

### Detailed Disclosure of Optional Embodiments

There is provided a method for high-throughput screening to determine cellular lifespan, the method comprising the steps of:
a) treating, in a well of a first multi-well plate, a sample containing cells with a condition that modulates lifespan of cells;
b) transferring a portion of the cells in the well of the first multi-well plate into a well of a second multi-well plate;
c) mixing, in a well of the second multi-well plate, the portion of cells with a fluorescent viability dye to stain the cells;
d) measuring fluorescence intensity of the stained cells in a plate reader to obtain a fluorescence intensity value;
e) measuring optical density of the cells in the plate reader to obtain an optical density value; and
f) normalizing the fluorescence intensity value with the optical density value, wherein the normalized fluorescence intensity value directly correlates with the viability of the cells.

The method may be for measuring the chronological lifespan of the cells, for screening anti-aging agents, for screening senolytic compounds, for screening anticancer agents, or for screening antifungal agents. The method may be for measuring the viability of cells.

The method may comprise a step (a) of treating the cells with a condition that modulates lifespan of cells, in a well of a multi-well plate.

Advantageously, the method may be used to screen the viability of cells following treatment with any kind of condition that may modulate the lifespan of cells.

The condition that modulates the lifespan of cells may be exposure to a lifespan modulating agent, or exposure to environmental conditions that modulate the lifespan of cells.

The lifespan modulating agent may be a chemical or biological reagent. The lifespan modulating agent may be selected from the group consisting of an anti-aging agent, anti-microbial agent, anti-fungal agent, anti-bacterial agent, anti-cancer agent, pharmaceutical drug, genotoxic agents, toxic chemical agent, and any mixture thereof.

The lifespan modulating agent may be an anti-aging agent. The anti-aging agent may be selected from the group consisting of rapamycin, metformin, resveratrol, caloric restriction, 2,5-anhydro-D-mannitol, aucubin, D-glucuronic acid, β-lapachone, isocytosine, rifamycin (sodium), crypotochlorogenic acid, isochlorogenic acid A, rhynchophylline, mupirocin. 3,5-dihydroxybenzoic acid, ethylparaben, nudifloramide, 4-methoxycinnamic acid, cefoselis (sulfate), DL-serine, avibactam (sodium), (S)-2-hydroxy-3-phenylpropanoic acid, vincristine (sulfate), N-acetyl-L-tyrosine, rubitecan, Royal Jelly acid, β-estradiol 17-acetate, 3,4-dimethoxycinnamic acid, 2-naphthol, N²,N²-dimethylguanosine, proxyphylline, amarogentin, lappaconitine (hydrobromide), 4-ethylphenol, purine, (S)-indoximod, actinonin, cirsimaritin, 6-biopterin, hederagenin, pentadecanoic acid, staurosporine, xanthoxylin, D-(+)-cellobiose, escin, 5α-cholestan-3-one, 20(S)-hydroxycholesterol, anthraquinone-2-carboxylic acid, L-kynurenine, quinine (hydrochloride dihydrate), DL-3-phenyllactic acid, retinoic acid, schisantherin B, narciclasine, doripenem (monohydrate), diflorasone, chlorogenic acid, glucosamine (hydrochloride), gardenoside, aloin B, 4-methylumbelliferone, lanolin, (R)-pyrrolidine-2-carboxylic acid, glycodeoxycholic acid (monohydrate), (−)-epigallocatechin, 2′-deoxyadenosine, wogonin, schisandrol B, 2,4-dihydroxybenzoic acid, hederacoside C, noricaritin, canrenone, glycoursodeoxycholic acid, glycodeoxycholic acid, cilastatin, emetine (dihydrochloride hydrate), norgestrel, heterophyllin B, hispidin, cinnamic acid, catechin, [6]-gingerol, epiberberine (chloride), terconazole, isoalantolactone, 2-furoic acid, vanillic acid, 5-acetylsalicylic acid, guggulsterone, thymol, homovanillic acid, uridine, gramicidin, gossypol, cyanidin-3-O-galactoside (chloride), kaempferol 3-O-β-D-glucuronide, L-homoserine, D-ribose (mixture of isomers), harmane, geraniol, 3,4-dimethoxyphenylacetic acid, 2-phenylpropionic acid, deltonin, 2-oxovaleric acid, estrone sulfate (potassium), (R)-citronellol, euphorbia factor L2, β-aminopropionitrile, bronopol, lincomycin (hydrochloride monohydrate), tridecanedioic acid, calcifediol, melanin, methyl acetylacetate, L-cysteine (hydrochloride hydrate), impulsin, formamide, 3-chloro-L-tyrosine, tetramethylpyrazine, esculin, DL-panthenol, baimaside, benzyl acetate, gentiopicroside, guaiazulene, engeletin, acivicin, tenofovir (hydrate), metyrosine, ripasudil, diphenmanil (methylsulfate), ambroxol (hydrochloride), VAL-083, aliskiren (hemifumarate), selenomethionine, cisatracurium (besylate), DL-methionine methylsulfonium (chloride), biapenem, dipotassium glycyrrhizinate, L-ornithine (hydrochloride), fosphenytoin (disodium), (R)-baclofen, trapidil, xanthinol nicotinate, cerivastatin (sodium), aprotinin, flavin adenine dinucleotide (disodium salt), biperiden (hydrochloride), streptomycin (sulfate), cefadroxil, diclofenac (sodium), estramustine (phosphate sodium), amifostine, quetiapine sulfoxide (dihydrochloride), minocycline (hydrochloride), L-selenomethionine, cangrelor (tetrasodium), chondroitin (sulfate), hyaluronidase, heparin (lithium salt), heparin (sodium salt) (MW 15 kDa), gentamicin (sulfate), polidocanol, plicamycin, nedaplatin, copanlisib dihydrochloride, hemin, 5-fluorouracil, methotrexate, atorvastatin and any mixture thereof.

The calorie restriction may be performed by restricting energy sources such as sugar. The sugar may be selected from the group consisting of sucrose, fructose, glucose and any mixture thereof.

The conditions that can modulate the lifespan of cells may include nutrients such as carbohydrates such as monosaccharides, disaccharides, oligosaccharides and polysaccharides, proteins such as contractile proteins, enzymes, hormonal proteins, structural proteins, storage proteins, antibodies and transport proteins and lipids such as fatty acids, triglycerides, phospholipids, sterols, sphingolipids, other alcohols, fatty aldehydes, and ketone bodies, hydrocarbons, lipid-soluble vitamins, and hormones, carbon, nitrogen, amino acids, phosphate, nucleosides, nucleotides, nutrient starvation, osmotic condition, oxidative stress, temperature, pH, oxygen concentration, and any mixture thereof.

The treating step (a) may be performed in a first multi-well plate, wherein the first multi-well plate of step (a) is different to the second multi-well plate of step (c).

The treating step (a) may comprise a step (a1) of exposing the cells to a condition that modulates lifespan of the cells.

The exposing step (a1) may comprise the step of treating the cells with a lifespan modulating agent.

The cells may be treated with the lifespan modulating agent at a range of various concentrations. The cells may be treated with the lifespan modulating agent at serially double-diluted concentrations. The cells may be treated with the lifespan modulating agent at a concentration in the range of about 0 nM to about 2 M, more than 0 nM to about 2 M, or about 0.0001 nM to about 2 nM. The cells may be treated with the lifespan modulating agent at a concentration in the range of 0 nM to about 2 M, 0 nM to about 0.0001 nM, 0 nM to about 0.001 nM, 0 nM to about 0.01 nM, 0 nM to about 0.1 nM, 0 nM to about 1 nM, 0 nM to about 10 nM, 0 nM to about 100 nM, 0 nM to about 1 μM, 0 nM to about 10 μM, 0 nM to about 100 μM, 0 nM to about 1 mM, 0 nM to about 10 mM, 0 nM to about 100 mM, 0 nM to about 1M, about 0.0001 nM to about 0.001 nM, about 0.0001 nM to about 0.01 nM, about 0.0001 nM to about 0.1 nM, about 0.0001 nM to about 1 nM, about 0.0001 nM to about 10 nM, about 0.0001 nM to about 100 nM, about 0.0001 nM to about 1 μM, about 0.0001 nM to about 10 μM, about 0.0001 nM to about 100 μM, about 0.0001 nM to about 1 mM, about 0.0001 nM to about 10 mM, about 0.0001 nM to about 100 mM, about 0.0001 nM to about 1M, about 0.0001 nM to about 2 M, about 1 μM to about 10 μM, about 1 μM to about 100 M, about 1 μM to about 1 mM, about 1 μM to about 10 mM, about 1 μM to about 100 mM, about 1 μM to about 1M, about 1 μM to about 2 M, about 10 UM to about 100 μM, about 10 μM to about 1 mM, about 10 μM to about 10 mM, about 10 UM to about 100 mM, about 10 μM to about 1M, about 10 μM to about 2 M, about 100 μM to about 1 mM, about 100 μM to about 10 mM, about 100 μM to about 100 mM, about 100 μM to about 1M, about 100 μM to about 2 M, about 1 mM to about 10 mM, about 1 mM to about 100 mM, about 1 mM to about 1M, about 1 mM to about 2 M, about 10 mM to about 100 mM, about 10 mM to about 1M, about 10 mM to about 2 M, about 100 mM to about 1M, about 100 mM to about 2 M or about 1 M to about 2 M.

The treating step (a) may further comprise a step (a2) of exposing the cells to a condition that modulates cellular lifespan for a various duration. The treating step (a) may further comprise a step (a2) of exposing the cells to a condition that modulates cellular lifespan for a duration in the range of about 12 hours to about 90 days, about 12 hours to about 24 hours, about 12 hours to about 48 hours, about 12 hours to about 72 hours, about 12 hours to about 7 days, about 12 hours to about 14 days, about 12 hours to about 21 days, about 12 hours to about 30 days, about 12 hours to about 60 days, about 24 hours to about 48 hours, about 24 hours to about 72 hours, about 24 hours to about 7 days, about 24 hours to about 14 days, about 24 hours to about 21 days, about 24 hours to about 30 days, about 24 hours to about 60 days, about 24 hours to about 90 days, about 48 hours to about 72 hours, about 48 hours to about 7 days, about 48 hours to about 14 days, about 48 hours to about 21 days, about 48 hours to about 30 days, about 48 hours to about 60 days, about 48 hours to about 90 days, about 72 hours to about 7 days, about 72 hours to about 14 days, about 72 hours to about 21 days, about 72 hours to about 30 days, about 72 hours to about 60 days, about 72 hours to about 90 days, about 7 days to about 14 days, about 7 days to about 21 days, about 7 days to about 30 days, about 7 days to about 60 days, about 7 days to about 90 days, about 14 days to about 21 days, about 14 days to about 30 days, about 14 days to about 60 days, about 14 days to about 90 days, about 21 days to about 30 days, about 21 days to about 60 days, about 21 days to about 90 days, about 30 days to about 40 days, about 40 days to about 50 days, about 50 days to about 60 days or about 60 days to about 90 days. The treating step (a) may further comprise a step (a2) of exposing the cells to a condition that modulates cellular lifespan for a duration of more than 12 hours, more than 24 hours, more than 72 hours, more than 7 days, more than 14 days, more than 21 days, more than 30 days, more than 60 days or more than 90 days.

The method may comprise a step (b) of transferring a portion of the cells in the well of the multi-well plate of step (a) into the well of a second multi-well plate.

The transferring step (b) may comprise the step (b1) of shaking the first multi-well plate to form a cell suspension. The cells in the first multi-well plate may be adherent to the bottom of the well in the first multi-well plate. The shaking step (b1) may cause the cells to detach from the bottom of the well of the first multi-well plate and create a cell suspension in the well of the first microwell plate.

The shaking step (b1) may be performed at a frequency in a range of about 800 rpm to about 1200 rpm, about 800 rpm to about 1000 rpm or about 1000 rpm to about 1200 rpm.

The shaking step (b1) may be performed for a duration in the range of about 10 seconds to about 15 seconds, about 10 seconds to about 12 seconds or about 12 seconds to about 15 seconds.

The shaking step (b1) may be performed using a thermomixer.

Advantageously, the shaking step (b1) may achieve relatively fast and uniform detachment of cells from the well of the first microwell plate. Further advantageously, unlike conventional methods for detaching cells done by up-and-down pipetting, the shaking step may result in less bubble formation that may compromise accurate transfer of the cells from the first microwell plate to the second microwell plate. Typically, when bubbles are formed during pipetting, it is necessary to wait for the bubbles to disappear, or to manually burst the bubbles before taking the next step, which is not conducive to high-throughput screening. By using shaking instead of up-and-down pipetting, cell detachment may be achieved faster and more efficiently. In addition, by using shaking instead of pipetting, it may circumvent the need to use multiple pipette tips, thereby contributing to cost reduction, the process may be safer and most importantly, the risk of inaccurate transfer of cells may be avoided.

The transferring step (b) may be performed by transferring the portion of the cells, in the form of the cell suspension, using a pipette.

The multi-well plate may be a 6-well plate. 12-well plate, 24-well plate, 48-well plate, 96-well plate, 384-well plate or a 1536-well plate.

The multi-well plate may be a 96-well plate. Advantageously, 96-well plates may facilitate screening of limited chemical or genome-wide deletion strains.

The multi-well plate may be a clear plate or an opaque-walled plate. The opaque-wall plate may have white walls or black walls.

Advantageously, the method may yield similar results regardless of whether a clear plate or a black-walled plate is used. As black-walled plates may be more expensive, the ability to use clear plates may facilitate more cost-effective high-throughput screening.

The sample containing the cells may comprise cell culture media or phosphate buffered saline.

If a 96-well plate is used, then the sample containing cells may comprise about 30 μL to about 250 μL of cell culture media or phosphate buffered saline. The volume of the cell culture media or phosphate buffered saline in the sample may be in the range of about 30 μL to about 60 μL, about 30 μL to about 90 μL, about 30 μL to about 120 μL, about 30 μL to about 150 μL, about 30 μL to about 200 μL, about 30 μL to about 250 μL, about 60 μL to about 90 μL, about 60 μL to about 120 μL, about 60 μL to about 150 μL, about 60 μL to about 200 μL, about 60 μL to about 250 μL, about 90 μL to about 120 μL, about 90 μL to about 150 μL, about 90 μL to about 200 μL, about 90 μL to about 250 μL, about 120 μL to about 150 μL, about 120 μL to about 200 μL, about 120 μL to about 250 μL, about 150 μL to about 200 μL, about 150 μL to about 250 μL, or about 200 μL to about 250 μL.

The cells may be fungal cells, bacterial cells or animal cells. The fungal cells may be yeast cells. The yeast cells may be cells of yeast selected from the group consisting of Saccharomyces cerevisiae, Saccharomyces pombe, Pichia pastoris and any mixture thereof.

The animal cells may be mammalian cells. The mammalian cells may be human cells. The human cells may be primary cells or immortal cells. The human cell may be a cancer cell. The human cell may be selected from the group consisting of a kidney cell, liver cell, muscle cell, lung cell, neuron, retinal cell, skin cell and any mixture thereof. The cells may be human embryonic kidney 293 (HEK293) cells or lung epithelial (A549 cells) or fibroblast (IMR90) cells.

Advantageously, the method may be used for fungal cells, bacterial cells and/or animal cells. Although there are critical differences in aging of yeast and animals, the method may advantageously be used in both yeast and animals, and in different cell types within each category.

The method may comprise a step (c) of mixing, in a well of the second multi-well plate, a sample containing the portion of cells with a fluorescent viability dye to stain the cells.

The mixing step (c) may comprise a step (c1) of shaking the second multi-well plate.

The shaking step (c1) may be performed at a frequency in a range of about 800 rpm to about 1200 rpm, about 800 rpm to about 1000 rpm or about 1000 rpm to about 1200 rpm.

The shaking step (c1) may be performed for a duration in the range of about 10 seconds to about 15 seconds, about 10 seconds to about 12 seconds or about 12 seconds to about 15 seconds.

The shaking step (c1) may be performed using a thermomixer.

Advantageously, the shaking step (c1) may achieve relatively fast and uniform mixing of the cells with the fluorescent viability dye. Further advantageously, unlike conventional methods for mixing done by up-and-down pipetting, the shaking step may result in less bubble formation that may compromise fluorescence and optical density reading. Bubbles that form in conventional pipetting techniques may skew the fluorescence and optical density readings and may give a false result. Typically, when bubbles are formed during pipetting, it is necessary to wait for the bubbles to disappear, or to manually burst the bubbles before taking any readings, which is not conducive to high-throughput screening. By using shaking instead of pipetting, the mixing may be performed faster and more efficiently. In addition, by using shaking instead of pipetting, it may circumvent the need to use multiple pipette tips, thereby contributing to cost reduction, the process may be safer and most importantly, the risk of an artificial or false reading may be avoided.

The fluorescent viability dye may quantitatively differentiate between dead cells and viable cells.

The fluorescent viability dye may emit fluorescence only when it is contacted with dead or damaged cells, or the fluorescent viability dye may emit fluorescence only when it is contacted with living cells.

The fluorescent viability dye may have excitation and emission wavelengths that do not affect optical density measurements.

The fluorescent viability dye may be selected from the group consisting of propidium iodide, PrestoBlue™, 3-(4,5-Dimethylthiazol-2-yl)-2,5-Diphenyltetrazolium Bromide (MTT), 2,3-Bis-(2-Methoxy-4-Nitro-5-Sulfophenyl)-2H-Tetrazolium-5-Carboxanilide (XTT), alamarBlue reagent, 7-hydroxy-10-oxidophenoxazin-10-ium-3-one (resazurin), SYTO 9, 2-chloro-4-(2,3-dihydro-3-methyl-(benzo-1,3-thiazol-2-yl)-methylidene)-1-phenylquinolinium iodide (Fun 1), disodium 2,2′-ethene-1,2-diylbis [5-({4-anilino-6-[bis(2-hydroxyethyl)amino]-1,3,5-triazin-2-yl}amino)benzenesulfonate] (Calcofluor White), 3′,6′-Bis(acetyloxy)spiro[isobenzofuran-1 (3H), 9′-[9H]xanthen]-3-one (fluorescein diacetate), N,N,N′,N′-Tetramethyl-3,6-acridinediamine (acridine orange), 2′-(4-Ethoxyphenyl)-5-(4-methyl-1-piperazinyl)-2,5′-bi-1H-benzimidazole trihydrochloride (Hoechst 33342), and any combination thereof.

The fluorescent viability dye may be propidium iodide. Propidium iodide (PI) is a nucleic acid intercalating fluorescent dye that only enters the dead cells and, thus, may be a powerful marker for direct quantification of cell viability. PI may fluoresce only when in contact with dead or damaged cells. It may therefore used to detect necrotic or apoptotic cells. It may be impermeable to live cells with intact membranes, but may be able to penetrate dead or cells with damaged membranes to bind to DNA and RNA. Upon intercalating between the bases, the quantum signal of PI increases drastically, which can then be detected by the microplate reader. Previously, a PI-based approach was used for measuring CLS and quantifying cell viability by analyzing the images acquired by a fluorescent cell counter, UV transilluminator, or flow cytometry. However, multiple sample processing, imaging, and software requirements for data analysis were expensive and time-consuming, which hindered these methods in high-throughput applications.

In the mixing step (c), the propidium iodide may be present at a concentration in the range of about 3 μg/mL to about 7 μg/mL in phosphate buffered saline. The concentration of propidium iodide may be in the range of about 3 μg/mL to about 4 μg/mL, about 3 μg/mL to about 5 μg/mL, about 3 μg/mL to about 6 μg/mL, about 4 μg/mL to about 5 μg/mL, about 4 μg/mL to about 6 μg/mL, about 4 μg/mL to about 7 μg/mL, about 5 μg/mL to about 6 μg/mL, about 5 μg/mL to about 7 μg/mL, or about 6 μg/mL to about 7 μg/mL.

The cells may be mixed with the propidium iodide for a duration in the range of about 10 minutes to about 20 minutes, about 10 minutes to about 15 minutes or about 15 minutes to about 20 minutes. The mixing step (c) may be performed in the dark.

The fluorescence of propidium iodide may be measured at an excitation wavelength of about 535 nm and emission wavelength of 617 nm.

The optical density may be measured at about 600 nm (OD₆₀₀). The optical density value may directly correlate with the number, density or concentration of cells.

The plate reader may be capable of measuring the absorbance, fluorescence, luminescence, time-resolved fluorescence, fluorescence polarization, light scattering and/or nephelometry of each well of a multi-well plate.

The method may comprise a step (x1) of washing the cells after the transfer step (b) and before the mixing step (c).

The method may comprise a step (x2) of washing the cells after the mixing step (c) and before the measuring step (d).

The washing steps (x1) and (x2) may independently comprise the steps of:
y1) centrifuging the multi-well plate to separate the cells from the cell culture media, phosphate buffered saline, residual fluorescent viability dye and any mixture thereof;
y2) removing the cell culture media, phosphate buffered saline, residual fluorescent viability dye and any mixture thereof from the cells; and
y3) resuspending the cells in phosphate buffered saline.

If a 96-well plate is used, then the resuspension step (y3) may be performed in phosphate buffered saline in a volume in the range of about 30 μL to about 200 μL of. The resuspension step (y3) may be performed in a volume of phosphate buffered saline in the range of about 30 μL to about 60 μL, about 30 μL to about 90 μL, about 30 μL to about 120 μL, about 30 μL to about 150 μL, about 60 μL to about 90 μL, about 60 μL to about 120 μL, about 60 μL to about 150 μL, about 60 μL to about 200 μL, about 90 μL to about 120 μL, about 90 μL to about 150 μL, about 90 μL to about 200 L, about 120 μL to about 150 μL, about 120 μL to about 200 μL, or about 150 μL to about 200 μL.

The washing steps (x1) and (x2) may independently be repeated multiple times. The washing steps (s1) and (x2) may independently be repeated 1, 2, 3, 4 or 5 times.

The method steps (c) to (f) may be performed within a duration of less than about 45 minutes, less than about 30 minutes, less than about 20 minutes. The method steps (c) to (f) may be performed within a duration in the range of about 5 minutes to about 20 minutes, about 5 minutes to about 30 minutes or about 5 minutes to about 45 minutes.

The method may further comprise a step (g) of comparing the normalized fluorescence intensity value of cells treated with the lifespan modulating condition with the fluorescence intensity value of positive control cells and negative control cells.

The fluorescence intensity value of the positive control cells may be measured by measuring the fluorescence intensity of a sample containing cells with a fluorescent viability dye to stain the cells, whereby:
the sample containing cells comprises all dead cells, if the fluorescent viability dye emits fluorescence only when it is contacted with dead or damaged cells, or
the sample containing cells comprises all live cells, if the fluorescent viability dye emits fluorescence only when it is contacted with living cells.

The fluorescence intensity of the negative control cells may be measured by measuring the fluorescence intensity of a sample containing cells without a fluorescent viability dye, whereby:
the sample containing cells comprises all dead cells, if the fluorescent viability dye emits fluorescence only when it is contacted with dead or damaged cells, or
the sample containing cells comprises all live cells, if the fluorescent viability dye emits fluorescence only when it is contacted with living cells.

The comparing step (g) may further comprise a step (g1) of calculating % cell survival.
% cell survival may be calculated using the following formula:

$$\% Survival = \frac{\frac{{I}_{ij}}{{OD}_{ij}} - \frac{{I}_{N}}{{OD}_{N}}}{\frac{{I}_{P}}{{OD}_{P}} - \frac{{I}_{N}}{{OD}_{N}}} \times 100 \%$$

wherein:
Iᵢⱼ is the fluorescence intensity measured at the well in the multi-well plate with plate coordinates i and j at the optimal excitation and emission wavelength of the fluorescent viability dye;
ODᵢⱼ is the optical density measured at the well in the multi-well plate with plate coordinates i and j;
I_{P} is the fluorescence intensity of the positive control cells stained with the fluorescent viability dye:
OD_{P} is the optical density of the positive control cells;
I_{N} is the fluorescence intensity of the negative control cells; and
OD_{N} is the optical density of the negative control cells.
% cell survival may be calculated using the following formula:

$$Survival = ( 1 - \frac{\frac{{I}_{ij} ( 535 nm / 617 nm )}{{OD}_{ij} ( 600 nm )} - \frac{{I}_{c} ( 535 nm / 617 nm )}{{OD}_{c} ( 600 nm )}}{\frac{{I}_{D} ( 535 nm / 617 nm )}{{OD}_{D} ( 600 nm )} - \frac{{I}_{c} ( 535 nm / 617 nm )}{{OD}_{c} ( 600 nm )}} ) 100 \%$$

where Iᵢⱼ (535 nm/617 nm) is the fluorescent intensity measured at the well with plate coordinates i and j with excitation at 535 nm and emission at 617 nm, I_{D} (535 nm/617 nm) is the fluorescent intensity measured for a cell with 100% dead cells stained with PI, I_{C} (535 nm/617 nm) is the fluorescent intensity measured for cells without PI, and the respective OD values are the absorption measured for the respective cells at 600 nm normalizing for the amount of cells.

The method may be for high-throughput screening of the viability of yeast cells following treatment with an anti-aging agent.

Advantageously, the method may be useful in screening numerous anti-aging agents to determine how effective the anti-aging agent is effective in improving cell viability or extending chronological lifespan.

Further advantageously, the method may circumvent the need to perform an outgrowth assay. Circumventing the need to perform an outgrowth assay may result in significant cost- and time-savings. In a typical assay for the viability of yeast cells following treatment with an anti-aging agent, the yeast cells would have to undergo an outgrowth assay on agar to manually count the colony forming units (CFU), which is not only more expensive but also time-consuming given that it must be performed over days before a result may be obtained. In conventional assays, even if fluorescence markers are used to determine cell viability, the results may have to be manually processed, and the results may only be qualitative. Therefore, conventional assays for viability of yeast cells may not be conducive for high-throughput screening. In contrast, the method as defined herein may circumvent the need for an outgrowth assay or manual analysis of the results, thereby allowing high-throughput screening of the viability of the cells in a quantitative manner and in a matter of minutes rather than days. In addition, by circumventing the need to perform an outgrowth assay, the method as defined herein may be able to detect the viability of the cells more accurately at the correct stage in the cell cycle.

If the method is for high-throughput screening of the viability of yeast cells following treatment with an anti-agent agent, the cell culture medium may be a YPD medium comprising 1% Bacto yeast extract, 2% Bacto peptone and 2% glucose or may be a synthetic defined (SD) medium contain 6.7 g/L yeast nitrogen base with ammonium sulfate without amino acids and 2% glucose or may be a synthetic defined (SD) medium contain 6.7 g/L yeast nitrogen base with ammonium sulfate and amino acids and 2% glucose or may be a synthetic defined (SD) medium contain 6.7 g/L yeast nitrogen base without ammonium sulfate and amino acids+CSM (complete supplement mixture) and 2% glucose.

In an example, there is provided a method for high-throughput screening of anti-aging agent in cells, the method comprising the steps of:
a) treating, in a well of a first multi-well plate, a sample containing cells with an anti-aging agent;
b) transferring a portion of the cells in the well of the first multi-well plate into a well of a second multi-well plate;
x1) optionally washing the cells;
c) mixing, in the well of the second multi-well plate, the portion of the cells with a fluorescent viability dye to stain the cells;
x2) optionally washing the stained cells;
d) measuring fluorescence intensity of the stained cells in a plate reader to obtain a fluorescence intensity value;
e) measuring optical density of the stained cells in the plate reader to obtain an optical density value;
f) normalizing the fluorescence intensity value with the optical density value, wherein the normalized fluorescence intensity value directly correlates with the viability of the cells;
g) comparing the normalized fluorescence intensity value of cells treated with the anti-aging agent with the fluorescence intensity value of positive control cells and negative control cells;
g1) calculating the % cell survival.

There is also provided a method for high-throughput screening to determine cellular lifespan, the method comprising the steps of:
a) plating, in a well of the multi-well plate, a sample containing cells and a fluorescent viability dye to stain the cells,
b) treating, in the well of the multi-well plate, the sample containing cells with a condition that modulates lifespan of cells; and
c) measuring fluorescence intensity of the stained cells in a plate reader to obtain a fluorescence intensity value.

If a 96-well plate is used, then the sample containing cells may comprise cells in a range of about 1,000 cells to about 200,000 cells, about 1,000 cells to about 2,000 cells, about 1.000 cells to about 10,000 cells, about 1,000 cells to about 25,000 cells, about 1,000 cells to about 50,000 cells, about 1,000 cells to about 75,000 cells, about 1,000 cells to about 100,000 cells, about 1.000 cells to about 150,000 cells, about 2,000 cells to about 10,000 cells, about 2,000 cells to about 25,000 cells, 2,000 cells to about 50,000 cells, 2,000 cells to about 75,000 cells, 2,000 cells to about 100,000 cells, 2,000 cells to about 150,000 cells, about 2,000 cells to about 200,000 cells, about 10,000 cells to about 25,000 cells, 10,000 cells to about 50,000 cells. 10,000 cells to about 75,000 cells, 10.000 cells to about 100,000 cells, 10,000 cells to about 150,000 cells, about 10,000 to about 200.000 cells, about 25.000 cells to about 50.000 cells, about 25.000 cells to about 75,000 cells, about 25,000 cells to about 100,000 cells, about 25,000 cells to about 150,000 cells, about 25,000 cells to about 200,000 cells, about 50,000 cells to about 75,000 cells, about 50,000 cells to about 100.000 cells, about 50,000 cells to about 150,000 cells, about 50,000 cells to about 200,000 cells, about 75,000 cells to about 100,000 cells, about 75,000 cells to about 150,000 cells, about 75,000 cells to about 200,000 cells, about 100,000 cells to about 150,000 cells, about 100,000 cells to about 200,000 cells or about 150,000 cells to about 200,000 cells.

### BRIEF DESCRIPTION OF DRAWINGS

The accompanying drawings illustrate a disclosed embodiment and explain the principles of the disclosed embodiment. However, the drawings are designed for illustration purposes only, and not as a definition of the limits of the invention.

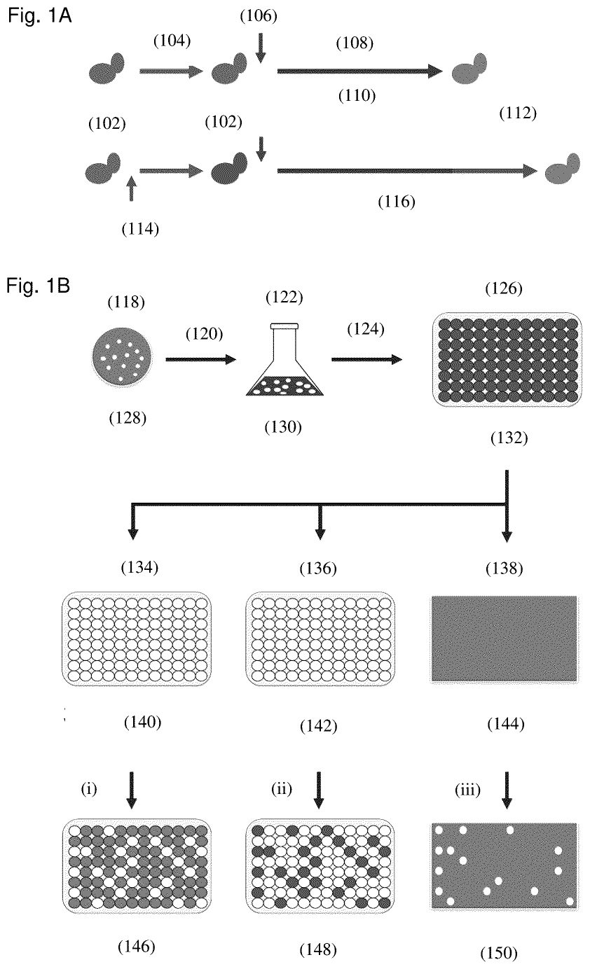

### FIG. 1.

FIG. 1 refers to a set of schematic diagrams describing methods to measure the chronological lifespan of the yeast. FIG. 1A refers to a schematic representation for determining the anti-aging compounds that extend the chronological lifespan of the yeast. (102) refers to live cell, (104) refers to growth phase, (106) refers to nutrient depletion, (108) refers to stationary phase, (110) refers to chronological lifespan. (112) refers to cell death, (114) refers to anti-aging compound and (116) refers to extension of chronological lifespan. FIG. 1B refers to a flowchart of propidium iodide (PI) fluorescence-based PICLS method, traditional outgrowth methods in YPD liquid medium and Yeast Extract-Peptone-Dextrose (YPD) agar medium (spotting assay) for screening the chemical agents to identify the anti-aging compounds. (118) refers to yeast. (120) refers to yeast cells inoculation. (122) refers to yeast culture. (124) refers to 0.2 OD600 nm yeast culture. (126) refers to chemical agents in 96-well plate, (128) refers to yeast cells grown onto YPD agar medium at 30° C. for 2 days, (130) refers to yeast cells grown in synthetic defined (SD) medium at 30° C. for 12 hours to 16 hours, (132) refers to yeast cells incubated with chemicals and grown in 200 μL SD medium, (134) refers to propidium iodide fluorescence method, (136) refers to outgrowth in liquid medium, (138) refers to outgrowth onto agar medium, (140) refers to an aliquot of cells stained with 5 μg/mL PI in a fresh 96-well plate at 30° C. for a duration of (i) 15 minutes, (142) refers to an aliquot of cells transferred into 200 μL. YPD medium in a fresh 96-well plate and incubated at 30° C. for a duration of (ii) 24 hours, (144) refers to an aliquot of cells spotted onto YPD agar medium and incubated at 30° C. for a duration of (iii) 48 hours. (146) refers to PI fluorescence reading at 535 nm excitation and 617 nm emission by microplate reader. (148) refers to cell outgrowth reading at OD600 nm by microplate reader and (150) refers to cell growth visualized by agar plate imaging.

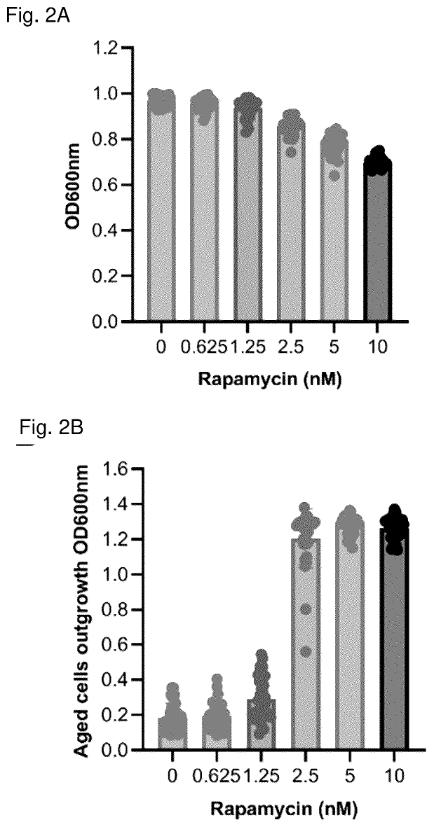

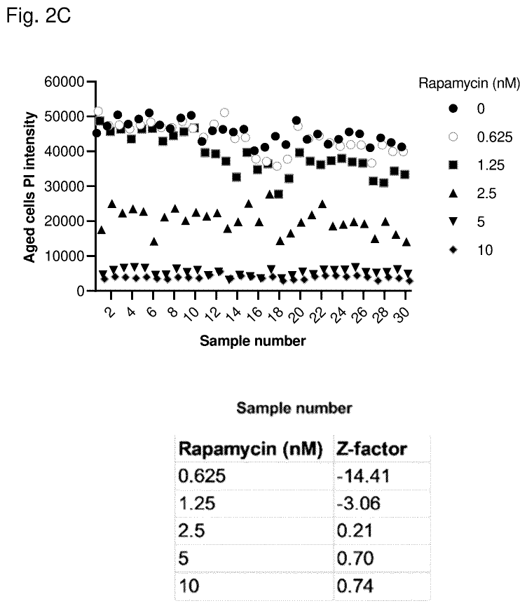

### FIG. 2.

FIG. 2 refers to a set of graphs showing the Z-factor analysis to evaluate of the quality of the high-throughput screening method. FIG. 2A refers to a graph of cell growth OD600 nm plotted against different concentrations of rapamycin. FIG. 2B refers to a graph of aged cell growth OD600 nm plotted against different concentrations of rapamycin. FIG. 2C refers to a plot of cell survival and subsequent derivation of Z-factor values.

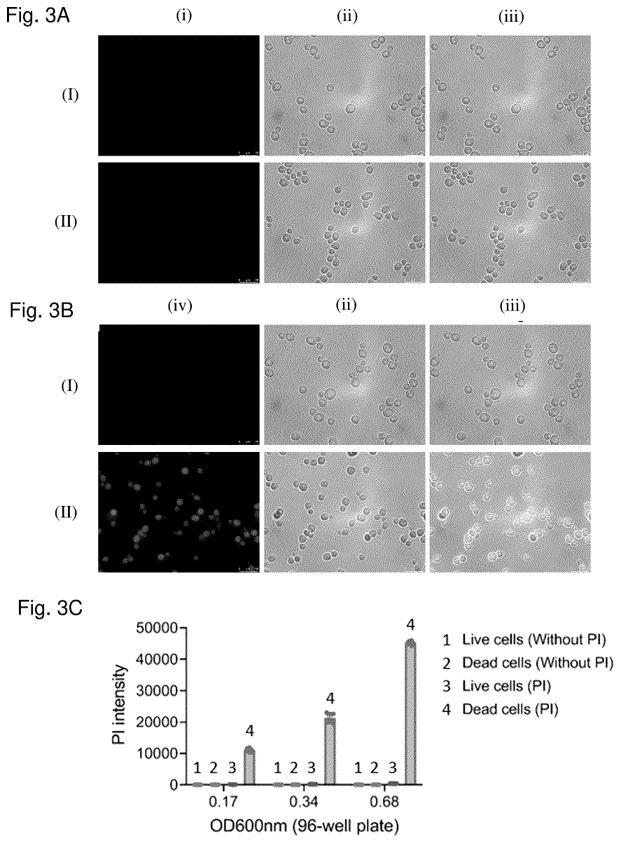

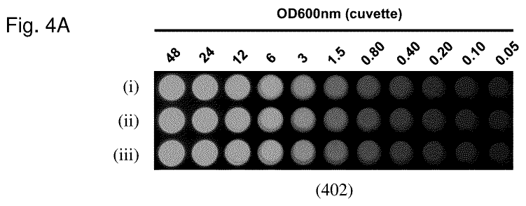

### FIG. 3.

FIG. 3 refers to set of images showing the propidium iodide staining analysis by microscopy and fluorescence intensity measuring using the microplate reader. FIG. 3A shows an image of non-stained PI exponentially grown yeast live cells (I) and boiled dead cells (II) being visualized under fluorescence microscopy. (i) indicates without PI, (ii) indicates DIC and (iii) indicates the merged image. FIG. 3B shows an image of PI stained (5 μg/ml) exponentially grown yeast live cells (I) and boiled dead cells (II) being visualized under fluorescence microscopy. (iv) indicates with PI, (ii) indicates DIC and (iii) indicates the merged image. FIG. 3C shows a graph of fluorescence intensity analysis for ten replicates of (1) live cells without PI staining, (2) dead cells without PI staining, (3) live cells with PI staining and (4) dead cells with PI staining, with different OD600 nm in the 96-well plate.

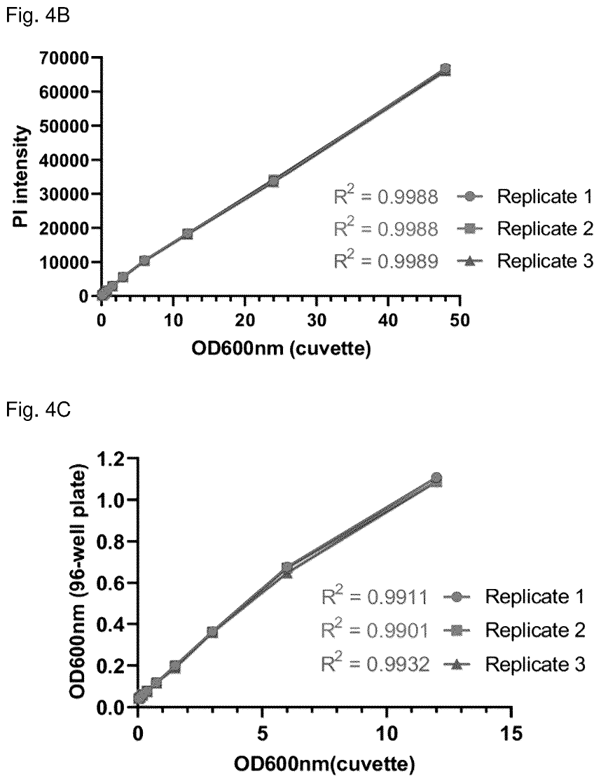

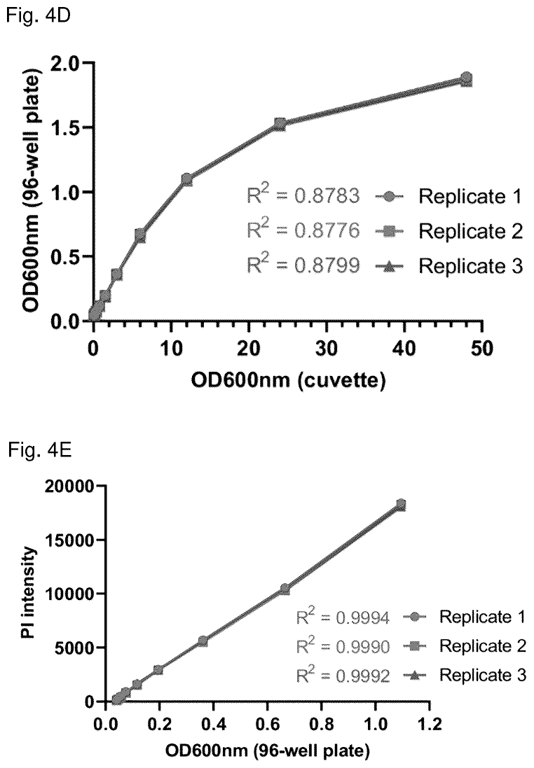

### FIG. 4.

FIG. 4 refers to a series of images describing the experiments performed to develop the propidium iodide fluorescent-based method for measuring the chronological lifespan. FIG. 4A shows an image of three replicates (i), (ii) and (iii) of diluted PI-stained yeast boiled cells (48 to 0.05 OD600 nm) in a black 96-well plate. The lighter colour indicates higher PI fluorescence. (402) refers to the yeast boiled dead cells stained with PI. FIG. 4B refers to a graph showing the correlation between PI fluorescence intensity and cell OD600 nm of a cuvette measurement. FIG. 4C refers to a graph showing the correlation between cell OD600 nm of a black 96-well plate measurement and a cuvette measurement within 12 to 0.05 OD600 nm. FIG. 4D refers to a graph showing the correlation between cell OD600 nm of the black 96-well plate and the cuvette within 48 to 0.05 OD600 nm. FIG. 4E refers to a graph showing the correlation between PI fluorescence intensity and cell OD600 nm of the black 96-well plate.

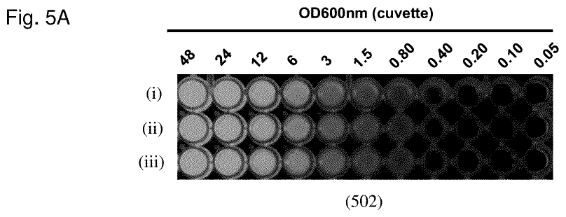

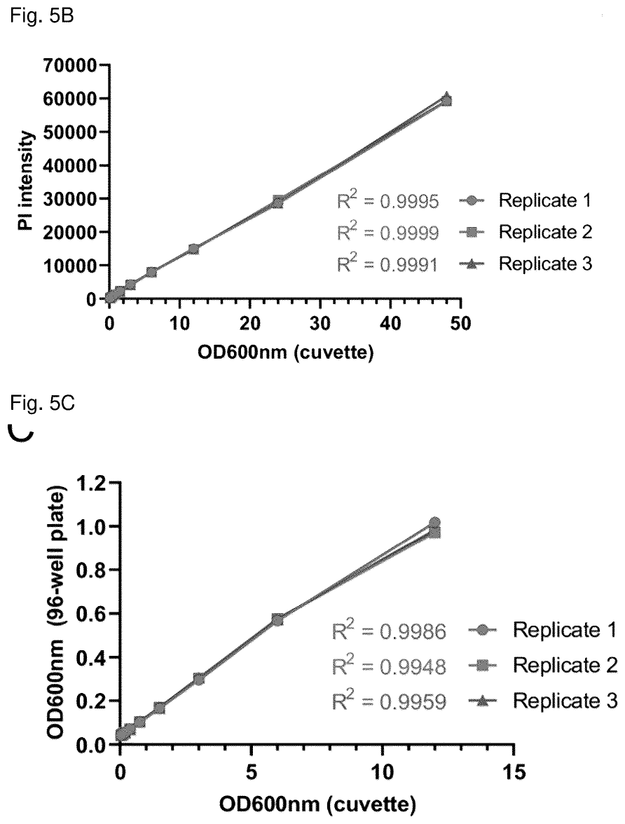

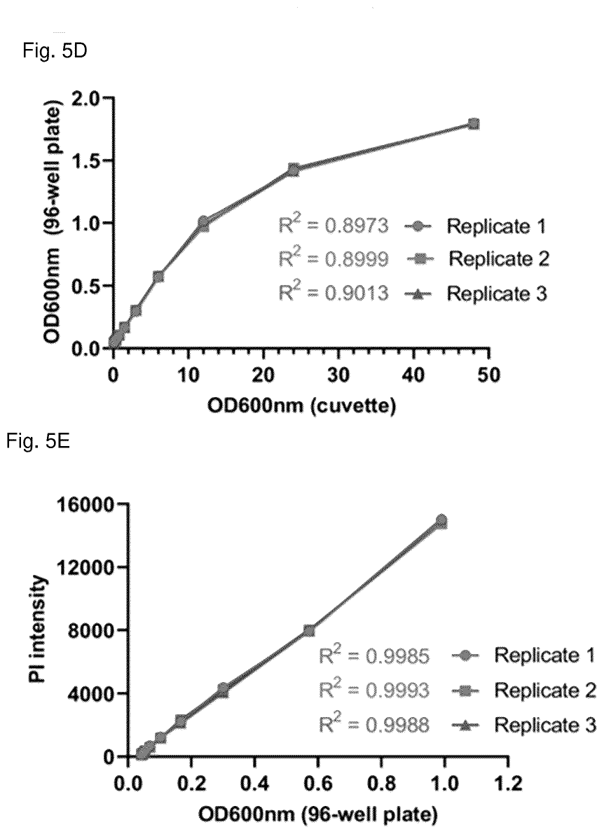

### FIG. 5.

FIG. 5 refers to a series of images describing the experiments performed to develop the propidium iodide fluorescent-based method for measuring the chronological lifespan in a clear 96-well plate. FIG. 5A shows an image of three replicates (i), (ii) and (iii) of diluted PI-stained yeast boiled cells (48 to 0.05 OD600 nm) in a clear 96-well plate. The lighter colour indicates higher PI fluorescence. (502) refers to the yeast boiled dead cells stained with PI. FIG. 5B refers to a graph showing the correlation between PI fluorescence intensity and cell OD600 nm of the cuvette. FIG. 5C refers to a graph showing the correlation between cell OD600 nm of the clear 96-well plate and the cuvette within 12 to 0.05 OD600 nm. FIG. 5D refers to a graph showing the correlation between cell OD600 nm of the clear 96-well plate and the cuvette within 48 to 0.05 OD600 nm. FIG. 5E refers to a graph showing the correlation between PI fluorescence intensity and cell OD600 nm of the clear 96-well plate.

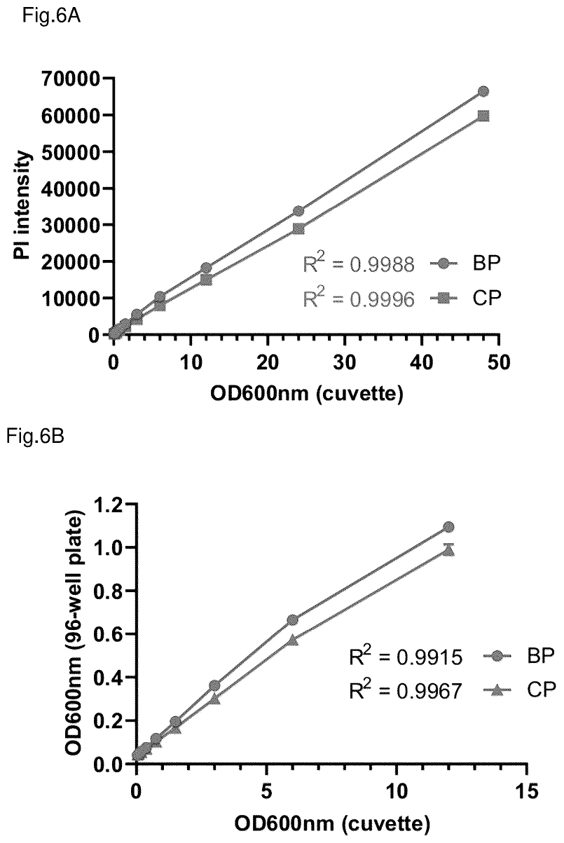

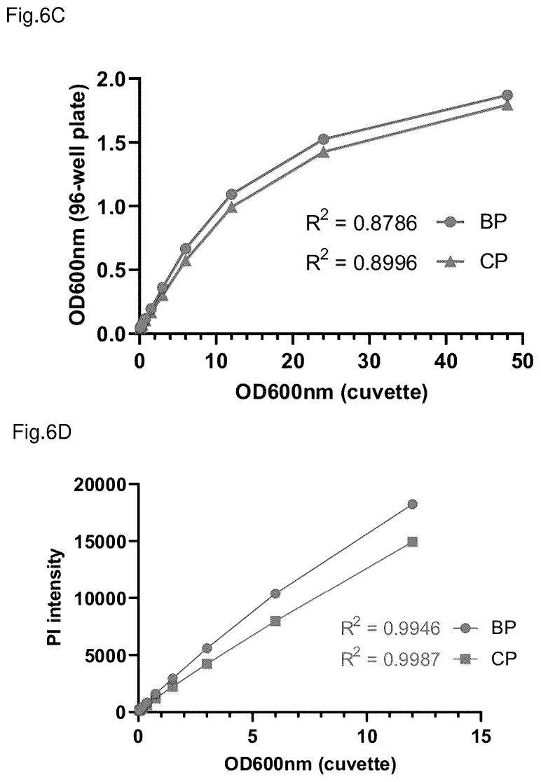

### FIG. 6.

FIG. 6 refers to a set of graphs showing the comparison of the propidium iodide fluorescence-based method for measuring the chronological lifespan in black and clear 96-well plates. FIG. 6A refers to a graph showing the comparison of the correlation between PI fluorescence intensity in black and clear 96-well plates and cell OD600 nm of the cuvette. FIG. 6B refers to a graph showing the comparison of the correlation between cell OD600 nm of the black and clear 96-well plate and the cuvette within 12 to 0.05 OD600 nm. FIG. 6C refers to a graph showing the correlation between cell OD600 nm of the black and clear 96-well plate and the cuvette within 48 to 0.05 OD600 nm. FIG. 6D refers to a graph showing the comparison of the correlation between PI fluorescence intensity and cell OD600 nm of the black and clear 96-well plate.

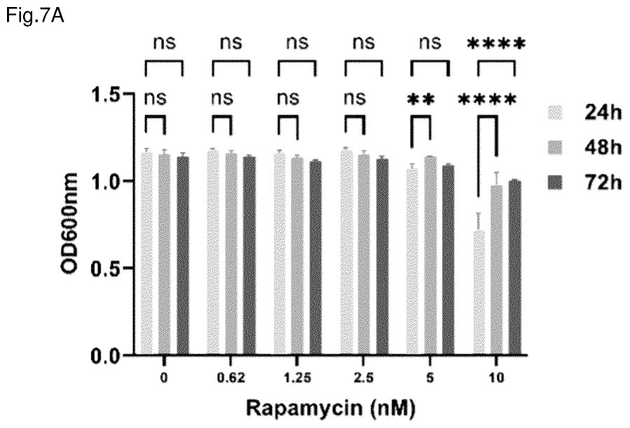

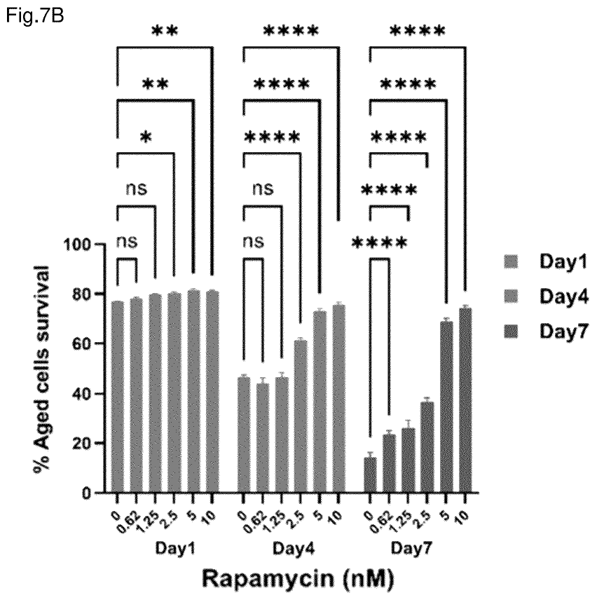

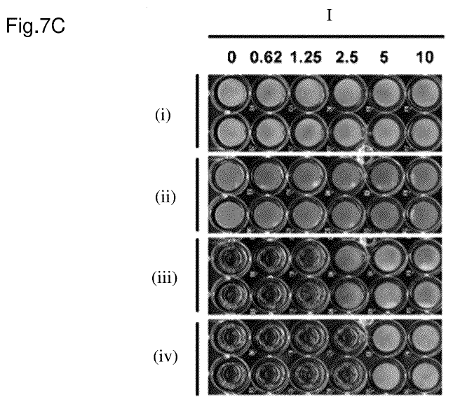

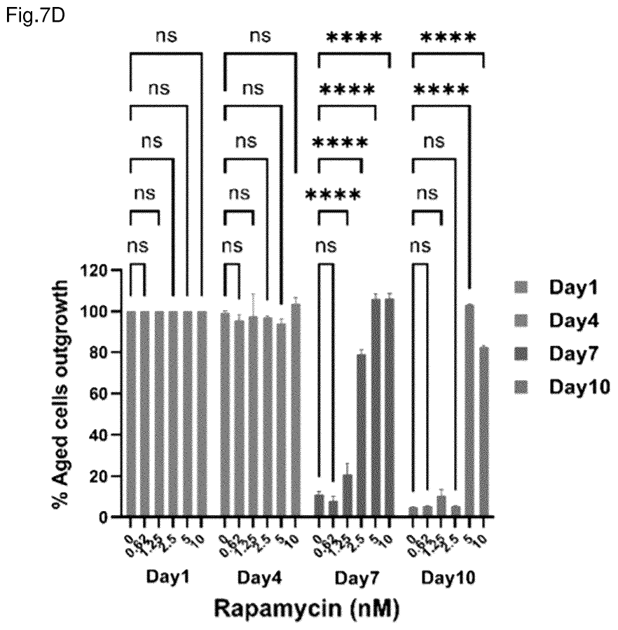

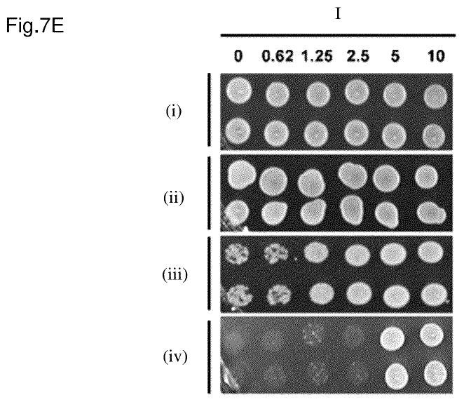

### FIG. 7.

FIG. 7 refers to a set of images describing a series of experiments performed to assess the effect of rapamycin on the chronological lifespan of yeast. FIG. 7A refers to a graph showing cell growth OD600 nm measured at time points 24 hours, 48 hours, and 72 hours, against different concentrations of rapamycin. FIG. 7B refers to a graph showing the chronological lifespan of cells incubated with different concentrations of rapamycin, recorded at different chronological age points, where growth time point 72 hours was considered as day 1. FIG. 7C refers to an image where the outgrowth method in Yeast Extract-Peptone-Dextrose (YPD) liquid medium was utilized to determine the CLS of the aged cells, and the growth time point 72 hours was considered as day 1. (I) indicates rapamycin, (i) indicates day 1, (ii) indicates day 4, (iii) indicates day 7 and (iv) indicates day 10. FIG. 7D refers to a graph showing outgrowth of different chronological age points incubated with different concentrations of rapamycin plotted relative to day 1 (72 hour time point). FIG. 7E refers to an image showing outgrowth on YPD agar plates at various chronological age points incubated with various concentrations of rapamycin (I). (i) indicates day 1, (ii) indicates day 4, (iii) indicates day 7 and (iv) indicates day 10.

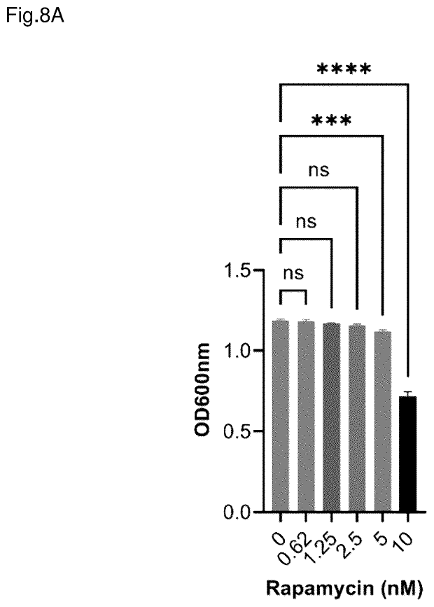

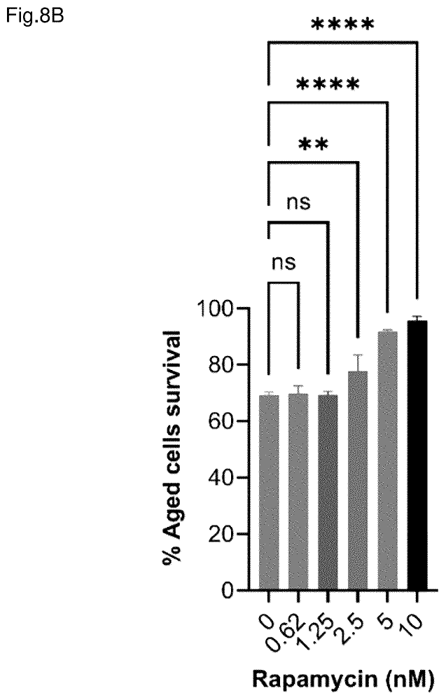

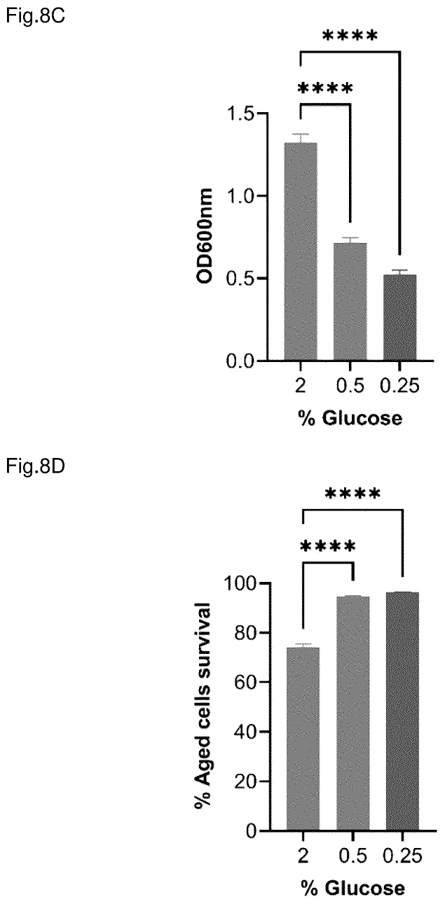

### FIG. 8.

FIG. 8 refers to a set of graphs which indicate that rapamycin and calorie restriction extend the CLS of yeast in the BY4743 strain, using the inventive protocol. FIG. 8A refers to a graph of cell growth OD600 nm plotted against different concentrations of rapamycin. FIG. 8B refers to a graph where aged cells survival was plotted against different concentrations of rapamycin. FIG. 8C refers to a graph of cell growth OD600 nm plotted against different concentrations of glucose. FIG. 8D refers to a graph where aged cells survival was plotted against different concentrations of glucose.

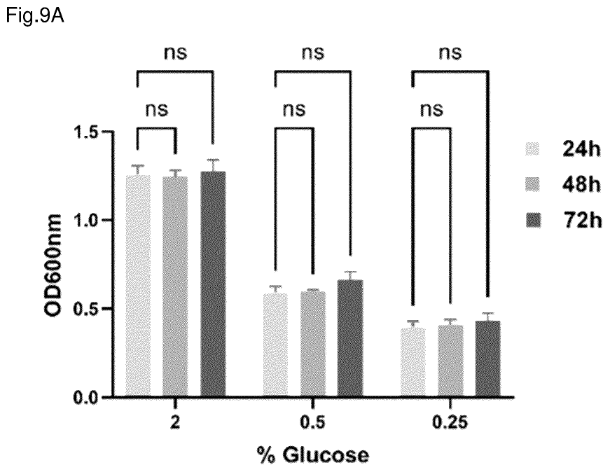

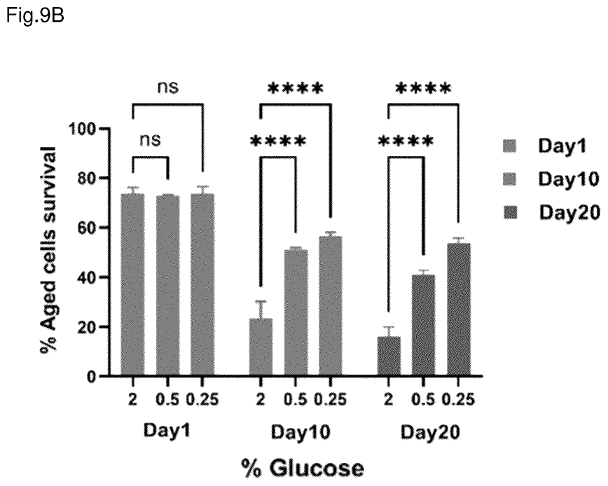

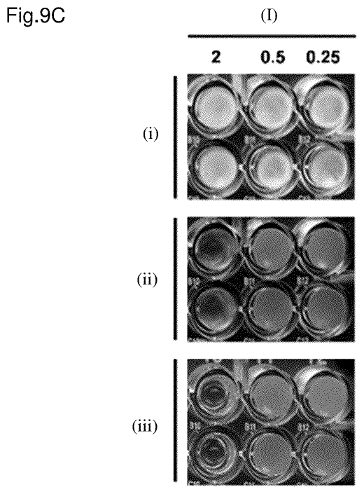

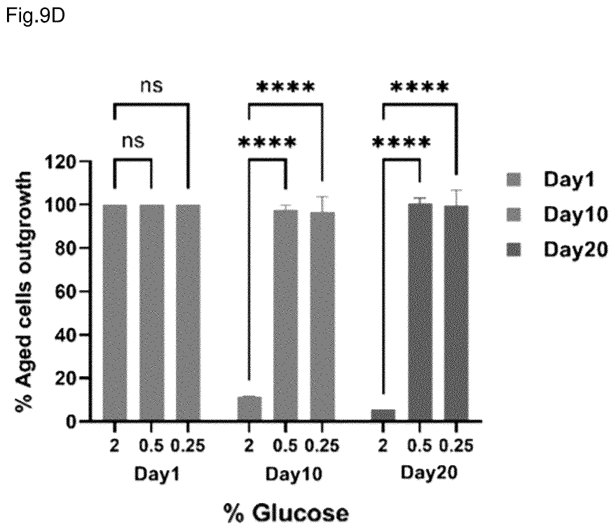

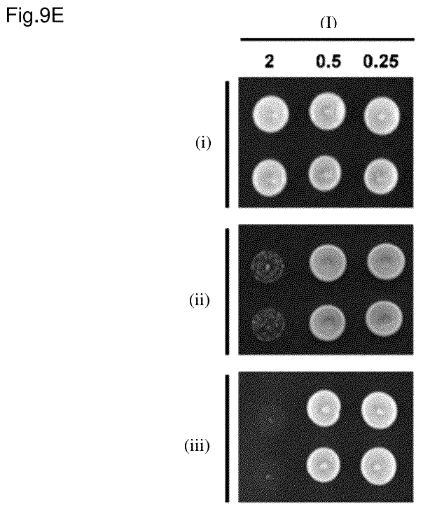

### FIG. 9.

FIG. 9 refers to a set of images describing a series of experiments performed to assess the effect of caloric restriction on the chronological lifespan of yeast. FIG. 9A refers to a graph showing cell OD600 nm measured at time points 24 hours, 48 hours, and 72 hours, against different concentrations of glucose. FIG. 9B refers to a graph showing the CLS of cells incubated with different concentrations of glucose, recorded at different chronological age points, where growth time point 72 hours was considered as day 1. FIG. 9C refers to an image where the outgrowth method in YPD liquid medium was utilized to determine the CLS of the aged cells, and the growth time point 72 hours was considered as day 1. (I) refers to the percentage of glucose in the medium, (i) indicates day 1, (ii) indicates day 10 and (iii) indicates day 20. FIG. 9D refers to a graph showing outgrowth of different chronological age points incubated with different concentrations of glucose plotted relative to day 1. FIG. 9E refers to an image showing outgrowth on YPD agar plates at various chronological age points incubated with various concentrations of glucose. (I) refers to the percentage of glucose in the medium, (i) indicates day 1, (ii) indicates day 10 and (iii) indicates day 20.

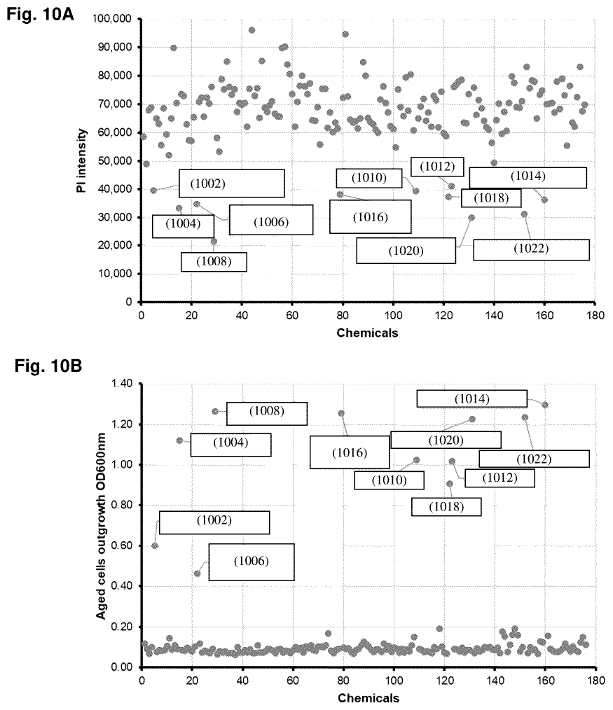

### FIG. 10

FIG. 10 refers to the high-throughput screening (HTS) outcome of various chemicals to identify novel anti-aging compounds. FIG. 10A refers to a graph evaluating the CLS of different chemical agents using the PI fluorescence-based method. Cell survival was quantified at chronological age point Day 7, and the growth time point 72 hours was considered as Day 1. (1002) refers to rifamycin (sodium), (1004) refers to DL-serine, (1006) refers to β-estradiol 17-acetate, (1008) refers to rapamycin, (1010) refers to melanin. (1012) refers to acivicin, (1014) refers to 2,5-anhydro-mannitol, (1016) refers to epiberberine (chloride), (1018) refers to engeletin, (1020) refers to selenomethionine, and (1022) refers to L-selenomethionine. FIG. 10B refers to a graph evaluating the CLS of aged cells using the outgrowth method in YPD liquid medium. The growth time point 72 hours was considered as Day 1. (1002) refers to rifamycin (sodium), (1004) refers to DL-serine, (1006) refers to β-estradiol 17-acetate, (1008) refers to rapamycin, (1010) refers to melanin, (1012) refers to acivicin, (1014) refers to 2,5-anhydromannitol, (1016) refers to epiberberine (chloride), 1018) refers to engeletin. (1020) refers to selenomethionine, and (1022) refers to L-selenomethionine.

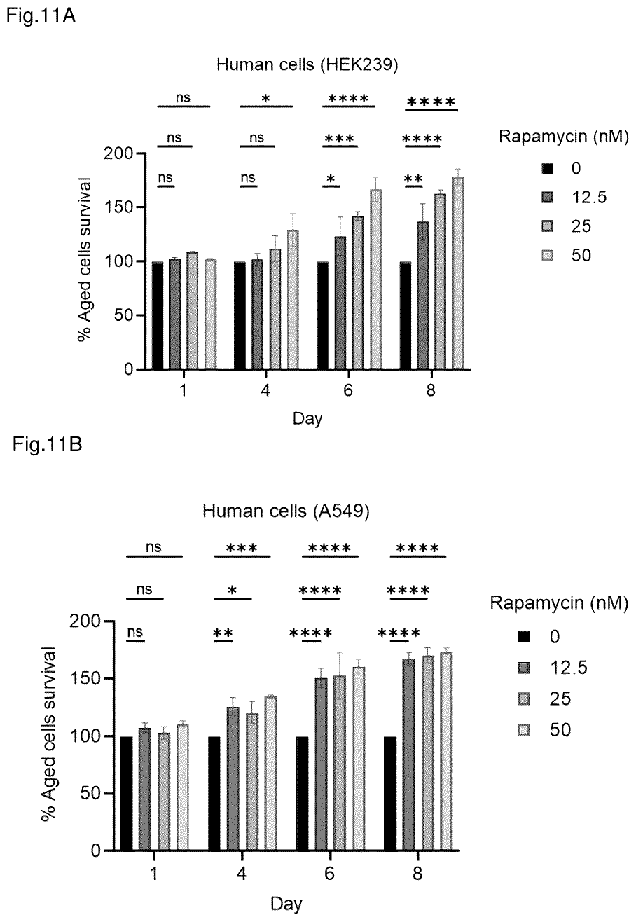

### FIG. 11

FIG. 11 refers to a set of graphs showing CLS experiments performed on human cell lines to test the validity of the PICLS method on human cells. FIG. 11A refers to a graph showing the aged cells survival of HEK293 cells at different chronological age points plotted against varying concentrations of rapamycin administered. FIG. 11B refers to a graph showing the aged cells survival of A549 cells at different chronological age points plotted against varying concentrations of rapamycin administered.

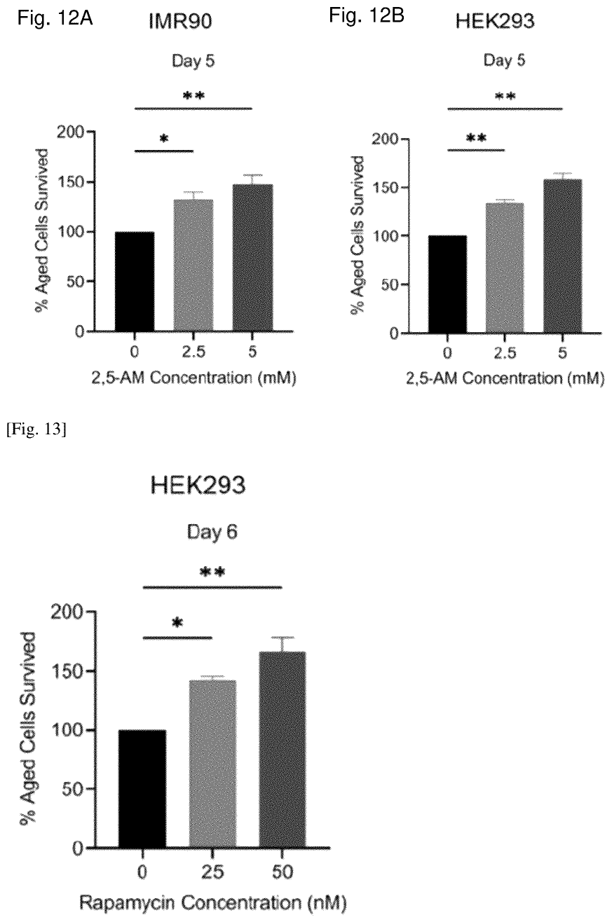

### FIG. 12

FIG. 12 refers to a set of graphs showing the outcome of the CLS determination using the inventive PI assay with 2,5-AM treatment. Cells were seeded in a 96-well plate with PI dissolved in the D10 medium. Fluorescence reading was taken on the sixth day after seeding. FIG. 12A is a graph showing the results for IMR90 cells and FIG. 12B is a graph showing the results for HEK293 cells.

### FIG. 13

FIG. 13 refers to a graph showing the CLS determination by cell viability assay with rapamycin treatment in HEK293 cells. Cells were seeded in a 96-well plate with PI dissolved in the D10 medium. Fluorescence reading was taken on the sixth day after seeding.

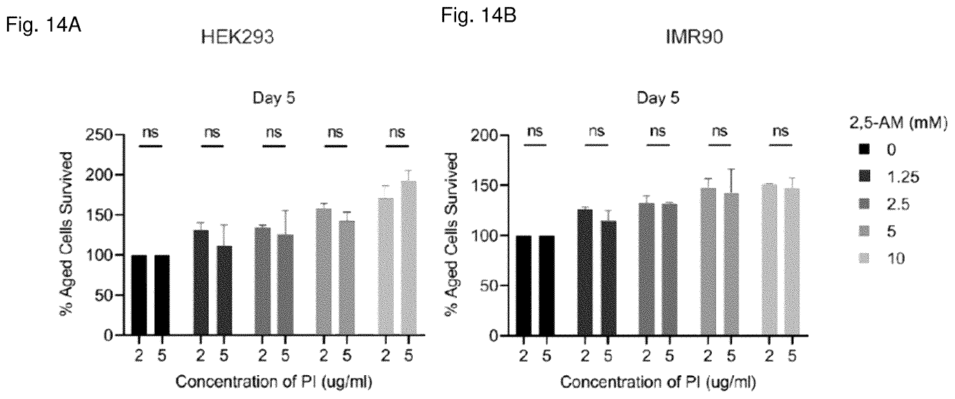

### FIG. 14

FIG. 14 refers to a set of graphs showing the cellular viability using the PI assay. FIG. 14A is a graph showing the cellular viability using 2 μg/mL PI or 5 μg/mL PI in HEK293 cells. FIG. 14B is a graph showing the cellular viability using 2 μg/mL PI or 5 μg/mL PI in IMR90 cells.

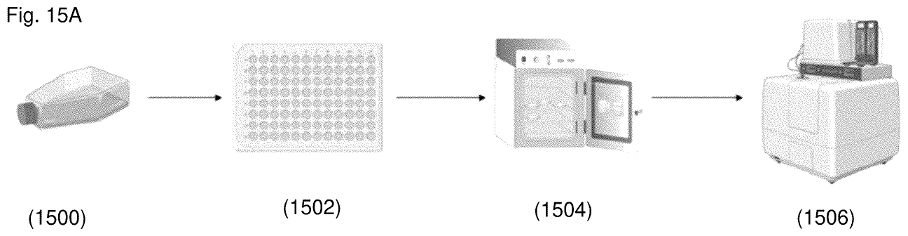

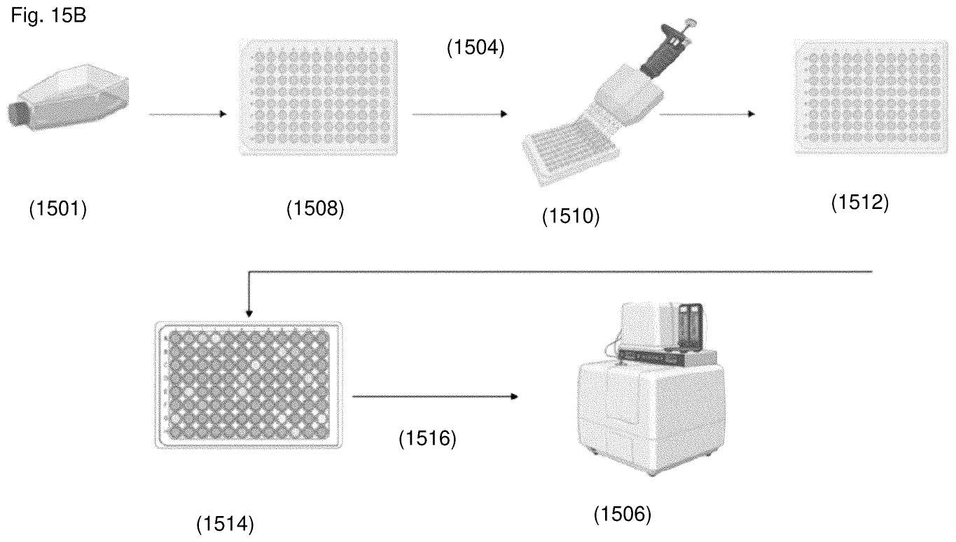

### FIG. 15

FIG. 15 refers to a set of schematic diagrams comparing the experimental procedures performed. FIG. 15A is a schematic diagram showing the experimental procedure of the inventive PI assay. FIG. 15B is a schematic diagram showing the experimental procedure of the PB outgrowth assay. (1500) refers to cell culture, (1502) refers to the step of seeding the cells in a 96-well plate with compounds and propidium iodide. (1504) refers to the step of incubating at 37° C. with 5% CO₂, (1506) refers to data collection via a microplate reader, (1508) refers to the step of seeding the cells in a 96-well plate with compounds, (1510) refers to the step of transferring the cells to a new plate. (1512) refers to the step of incubating for 1 to 2 days at 37° C. with 5% CO₂. (1514) refers to the PB assay, and (1516) refers to the step of incubating for 3 to 4 hours at 37° C. with 5% CO₂.

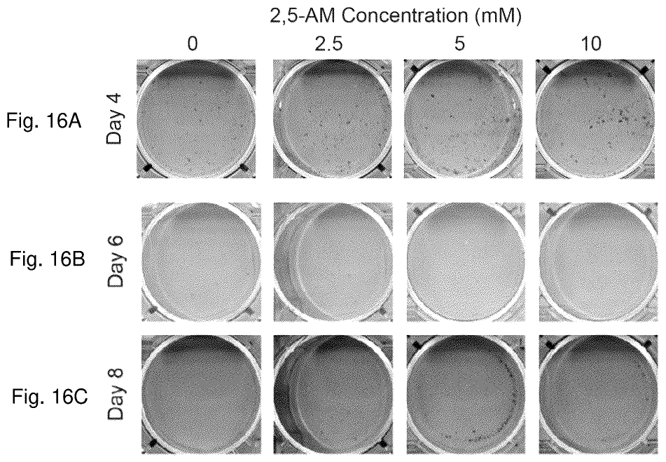

### FIG. 16

FIG. 16 refers to a set of photographic images showing the CLS determination by qualitatively assessing the ability of the cells to proliferate. Cells were plated without (control) or with 2,5-AM compound treatment at several concentrations. On the designated day after the cells were seeded and treated, cells were trypsinized and 2% (4 μL out of 200 μL) of the cells were transferred to a 6-well assay plate with fresh D10 medium. The assay plate was incubated at 37° C. in an incubator with 5% CO₂ for seven days before staining with the Crystal Violet Assay. FIG. 16A shows the photographic image of the assay result after Day 4. FIG. 16B shows the photographic image of the assay result after Day 6. FIG. 16C shows the photographic image of the assay result after Day 8.

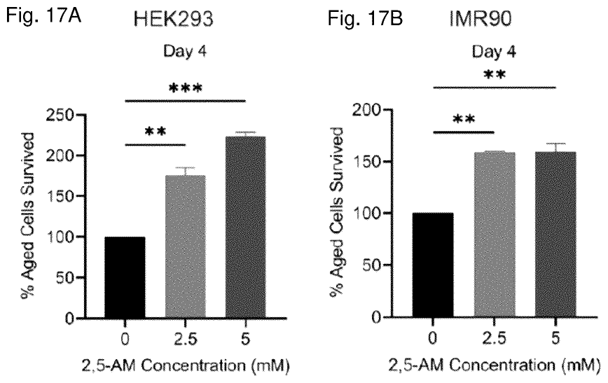

### FIG. 17

FIG. 17 refers to a set of graphs showing the outcome of the CLS determination using the outgrowth assay with 2,5-AM treatment. Cells were seeded in a 96-well plate with PI dissolved in the D10 medium. Fluorescence reading was taken on the sixth day after seeding. FIG. 17A is a graph showing the results for IMR90 cells and FIG. 17B is a graph showing the results for HEK293 cells.

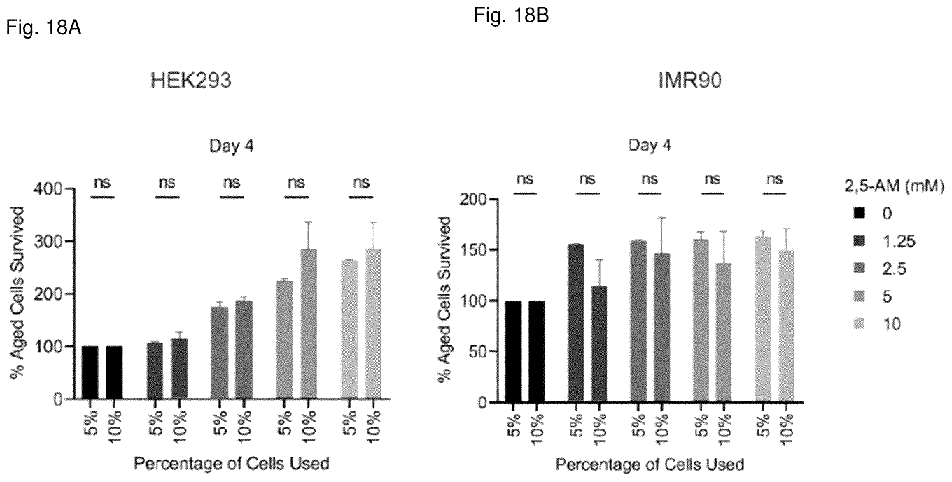

### FIG. 18

FIG. 18 refers to a set of graphs showing the CLS determination by quantitatively assessing the ability of the cells to proliferate with 2,5-AM treatment. FIG. 18A is a graph showing the cellular outgrowth for 5% (10 μL) and 10% (20 μL) of cells transferred to the assay plate in HEK293 cells. FIG. 18B is a graph showing the cellular outgrowth for 5% (10 μL) and 10% (20 μL) of cells transferred to the assay plate in IMR90 cells.

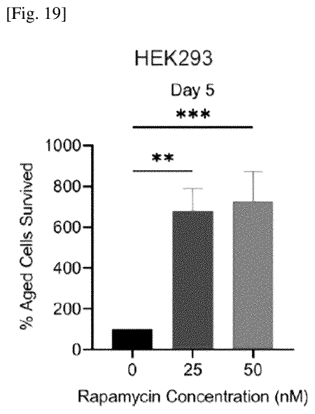

### FIG. 19

FIG. 19 refers to graph showing the CLS determination by outgrowth assay with rapamycin treatment in HEK293 cells. Cells were seeded in a 96-well plate with PI dissolved in the D10 medium. Fluorescence reading was taken on the sixth day after seeding.

### EXAMPLES

Non-limiting examples of the invention will be further described in greater detail by reference to specific Examples, which should not be construed as in any way limiting the scope of the invention.

### Materials

The prototrophic wild-type Saccharomyces cerevisiae CEN.PK113-7D yeast strain genetic background (Euroscarf, Köhlerweg, Oberursel, Germany), and the Saccharomyces cerevisiae BY4743 strain (Euroscarf. Köhlerweg, Oberursel. Germany) were used. The CEN.PK113-7D snf1Δ deletion strain was constructed. Standard rich Yeast Extract-Peptone-Dextrose (YPD) medium (containing 1% w/v Bacto yeast extract, 2% w/v Bacto peptone, and 2% w/v glucose) (BD, Franklin Lakes, New Jersey, United States of America) YPD agar (with 2.5% w/v Bacto agar) (BD, Franklin Lakes, New Jersey, United States of America), and synthetic defined (SD) medium (BD, Franklin Lakes, New Jersey, United States of America) contained 6.7 g/L yeast nitrogen base with ammonium sulfate without amino acids from BD DIFCO™ (BD, Franklin Lakes, New Jersey, United States of America) and 2% w/v glucose. Rapamycin (Enzo, Farmingdale, New York, United States of America) stock solution was prepared in dimethyl sulfoxide (DMSO) (Sigma-Aldrich, St. Louis, Missouri, United States of America). The maximum amount of DMSO used in any assay was 1%. 2,5-anhydro-D-mannitol (Santa Cruz Biotechnology, Texas, United States) and D-glucose (Sigma-Aldrich, St. Louis, Missouri, USA) were freshly prepared by direct dissolution of the compound in medium and further filtered under sterilized conditions.

The various compounds tested in Table 1 were obtained from Sigma-Aldrich (St. Louis, Missouri, United States of America), MedChemExpress (Monmouth Junction, New Jersey, United States of America), Enzo Life Sciences, Inc. (Farmingdale, New York, United States of America), Santa Cruz Biotechnology (Dallas, Texas, United States of America), or Biosynth Carbosynth (Compton, Berkshire, United Kingdom).

### Methods

### Yeast Cell Culture

Yeast strain was recovered from frozen glycerol stock at 30° C. on YPD agar medium. Yeast was grown in SD medium overnight at 30° C. with shaking at 220 revolutions per minute (rpm). Cells grown overnight were diluted to OD600 nm˜0.2 in fresh SD medium to initiate the chronological lifespan experiments.

### Chronological Lifespan Analysis in Yeast

Chronological lifespan (CLS) experiments were conducted in 96-well plates with 200 μL yeast culture. The prototrophic wild-type yeast strain (CEN.PK113-7D) and snf1Δ deletion strains were grown in the SD medium in 96-well plates at 30° C. Saccharomyces cerevisiae BY4743 strain was grown in the SD medium supplemented with histidine (40 mg/L), leucine (160 mg/L), and uracil (40 mg/L) in 96-well plates at 30° C. For assays testing the effect of rapamycin, cellular inoculum was transferred into 96-well plates containing serially double-diluted concentrations (0 to 10 nM) of rapamycin. For calorie restriction assay, cellular inoculums were prepared in SD medium containing 2% w/v, 0.5% w/v, and 0.25% w/v glucose and transferred to 96-well plates. Likewise, cellular inoculum was transferred into the 96-well plates containing serially double-diluted concentrations (0-8 mM) of D-fructose, D-mannitol, D-maltose, and D-sorbitol. Cells were incubated at 30° C., and the growth was measured at different time points. The growth time 72 hours was considered as day 1 for the CLS assay. Cell survival was quantified at various age time points by three different approaches: (i) propidium iodide fluorescence-based method, (ii) outgrowth in YPD liquid medium and (iii) spotting assay (FIG. 1).

### Propidium Iodide Fluorescence-Based Method in Yeast

40 μL yeast cells on different age time points were transferred into a 96-well plate different to the 96-well plate in which the CLS analysis was performed. Cells were washed and incubated in 100 μL 1×PBS with PI (5 μg/mL) for 15 minutes in the dark. Positive and negative sample control were included for quantitative analysis. Positive control (cells boiled at 100° C. for 15 min) was PI-stained and processed in the same 96-well plate. Samples without PI-stained cells served as the negative control. After incubation, cells were washed and resuspended in 100 μL PBS. The fluorescence reading of the samples (excitation at 535 nm, emission at 617 nm) and OD600 nm were measured by a microplate reader (BioTek, Winooski, Vermont, United States). The fluorescence intensity of each sample was normalized with OD600 nm. The normalized fluorescence intensity of each sample was subtracted from the background signal of the unstained negative sample. The obtained fluorescence intensity of the positive control sample (boiled dead cells) was considered 0% cell survival. Cell death was confirmed by allowing the boiled dead cells to grow in the medium. Cell survival of different age time point samples were calculated by normalizing the fluorescence intensity with positive control sample (boiled cells).

Hence, cell survival was calculated using the formula:

$$Survival = ( 1 - \frac{\frac{{I}_{ij} ( 535 nm / 617 nm )}{{OD}_{ij} ( 600 nm )} - \frac{{I}_{c} ( 535 nm / 617 nm )}{{OD}_{c} ( 600 nm )}}{\frac{{I}_{D} ( 535 nm / 617 nm )}{{OD}_{D} ( 600 nm )} - \frac{{I}_{c} ( 535 nm / 617 nm )}{{OD}_{c} ( 600 nm )}} ) 100 \%$$

where Iᵢⱼ (535 nm/617 nm) is the fluorescence intensity measured for each microplate well with plate coordinates © and j with excitation at 535 nm and emission at 617 nm, respectively, I_{D} (535 nm/617 nm) is the fluorescence intensity measured for a well with 100% dead cells stained with PI. I_{C} (535 nm/617 nm) is the fluorescence intensity recorded for cells without PI, and the respective OD values are the absorption measured for the respective cells at 600 nm normalizing for the amount of cells.

### Yeast Outgrowth in YPD Liquid Medium

Yeast stationary culture (3 μL) of different age time points were transferred to a second 96-well plate containing 200 μL YPD medium and incubated for 24 hours at 30° C. Outgrowth (OD600 nm) of aged cells was measured by the microplate reader. Quantification of cell survival for each age point was determined relative to day 1 (considered 100% cell survival). This quantification was performed by using the formula,

$$Cell survival on Day n = \frac{{Outgrowth}_{Day n}}{{Outgrowth}_{Day 1}} \times 100$$

where Outgrowth_{Day n} refers to the OD600 nm absorbance reading recorded on a specific day, Outgrowth Day 1 refers to the OD600 nm absorbance reading recorded on the first day where 100% cell survival is assumed.

### Yeast Spotting Assay

Yeast stationary culture (3 μL) of different age time points were spotted onto the YPD agar plate and incubated for 48 hours at 30° C. The outgrowth of aged cells on the YPD agar plate was photographed using the BioRad GelDoc imaging system (Bio-Rad, Hercules, California, United States).

### Z-Factor Analysis to Evaluate the Quality of High-Throughput Screening Method

The prototrophic yeast strain (CEN.PK113-7D) was grown in the synthetic defined medium with indicated concentrations of rapamycin in 96-well plates at 30° C. A total of 30 samples of individual rapamycin concentration was tested with DMSO control (0 nM rapamycin) for Z-factor analysis. Cell growth OD600 nm was measured at 72 hours using a microplate reader and a graph is plotted against different concentrations of rapamycin (FIG. 2A). The chronological lifespan of the aged cells was determined by the outgrowth method in YPD liquid medium. The growth time point 72 hours was considered as day 1. At chronological age point day 4, 3 μL culture were transferred to a second 96-well plate containing 200 μL YPD medium. Outgrowth OD600 nm in YPD liquid medium was measured after incubation for 24 hours at 30° C. using a microplate reader (FIG. 2B). The CLS of different concentrations of rapamycin incubated cells was determined using the propidium iodide fluorescence-based method (FIG. 2C). Cell survival at age point day 4 was quantified, and the growth time point 72 hours was considered day 1. Z-factor of different concentrations of rapamycin was analyzed using the formula Z=1−[3×(standard deviation of control sample+standard deviation of rapamycin sample)/(mean of control sample−mean of rapamycin sample)].

### High-Throughput Screening

High-throughput screening of various chemicals was tested using the same protocol for the propidium iodide fluorescence-based method as described above. The prototrophic yeast strain (CEN.PK113-7D) was grown in the synthetic defined medium with different chemical reagents with a single testing concentration (Table 1) in addition to eight replicates of DMSO and water controls in 96-well plates at 30° C.

Cell survival at age point day 7 was quantified, and the growth time point 72 hours was considered day 1 (FIG. 10A).

The CLS of the aged cells was further determined by the yeast outgrowth in YPD liquid medium method as described above. The growth time point 72 hours was considered as day 1. At chronological age point day 14, 3-μL of culture were transferred to a second 96-well plate containing 200 μL YPD medium. Outgrowth OD600 nm in YPD liquid medium was measured after incubation for 24 hours at 30° C. using a microplate reader (FIG. 10B).

### Human Cell Culture

Human embryonic kidney 293 (HEK293) cells (ATCC, Manassas, Virginia, United States of America), lung epithelial (A549) cells (ATCC, Manassas, Virginia, United States of America) and fibroblast (IMR90) cells (Coriell Institute, Camden, New Jersey, United States of America) were cultured in the standard D10 medium, consisting of high-glucose DMEM (HyClone, Cytiva, Marlborough, Massachusetts, USA) supplemented with 10% Fetal Bovine Serum (FBS) (Gibco™, ThermoFisher Scientific, Waltham, Massachusetts, USA) and 1% Penicillin Streptomycin Solution (Gibco™, ThermoFisher Scientific, Waltham, Massachusetts, USA). All cells were cultured in a humidified incubator with 5% CO₂ at 37° C.

### Chemical Treatment of Human Cell Culture

The stock solution of rapamycin was prepared in dimethyl sulfoxide (DMSO), followed by serial dilution to form various concentrations used in the experiment. The final DMSO concentration was kept at 0.05% in all experiments. The stock solution of 2,5-anhydro-D-mannitol was prepared in D10 medium. The chemical library used in screening were tested at 1 μM concentrations. Controls used were the same as the solvent applied, DMSO or water.

### Chronological Lifespan Analysis in Human Cells Using Crystal Violet

80,000 cells were seeded in a 96-well culture plate with or without compound treatment. On Day 4, Day 6 and Day 8 after seeding, floating dead cells were aspirated with exhausted D10 medium. Then, attached cells were gently washed with 1× Phosphate-buffered saline (PBS) to remove D10 medium residue and treated with 0.2 mL of 0.25% trypsin. Cells were gently mixed by pipetting and a 2% aliquot (4 μL) was transferred to 6-well experiment plates with fresh D10 medium. After seven days of growth in a 37° C. incubator with 5% CO₂, the crystal violet (CV) assay was performed on the test plate to determine the viability of cultured cells. For the CV assay, the media was first aspirated, before the cells were stained with 0.05% w/v CV and left to shake with 20 oscillation speed for 20 minutes. The media with CV was subsequently aspirated and the cells were washed once with water. The water was then aspirated and the plate inverted. Residual water was removed by tapping the plate dry and the plate was subsequently left to dry in air for at least 2 hours. Once dry, the plates were imaged using a scanner (Perfection V700 photo, Epson, Singapore).

### Chronological Lifespan Analysis in Human Cells Using PrestoBlue™ Cell Viability Reagent

The seeding, washing and trypsinization process was performed similarly to that for the crystal violet assay. 80.000 cells were seeded in a 96-well culture plate with or without compound treatment. At different age time points after seeding, floating dead cells were aspirated with exhausted D10 medium, cells were gently washed with 1×PBS to remove D10 medium residue and treated with 0.2 mL of 0.25% trypsin. Cells were gently mixed by pipetting and a 5% aliquot (10 μL) or 10% aliquot (20 μL) were transferred to 96-well experiment plates with fresh D10 medium. After one day of growth in a 37° C. incubator with 5% CO₂, the cell number was assessed with PrestoBlue™ Cell Viability Reagent (Invitrogen™, Waltham, Massachusetts, USA) according to the manufacturer's protocol. In brief, the cell viability reagent was diluted 10-fold with growth D10 medium. The D10 medium was aspirated from the 96-well plate and replaced with the diluted PrestoBlue reagent. After incubation in a 37° C. incubator with 5% CO₂ for 3 to 4 hours, reading was taken with a Synergy MX microplate reader at 560 nm excitation and 590 nm emission (BioTek, Winooski, Vermont, United States).

### Chronological Lifespan Analysis in Human Cells Using Propidium Iodide Fluorescence-Based Method

The assay was performed by preparing the cells with PI (2 μg/mL or 5 μg/mL) in a D10 medium. 200 μL culture (8×10⁴ cells) was seeded per well in a 96-well plate with different concentrations of the anti-aging drug (rapamycin or 2,5-AM) and no treatment control (DMSO). The culture plate was kept away from light throughout the experiment and incubated at 37° C. with 5% CO₂. At different age time points, reading was taken with a Synergy MX microplate reader at 535 nm excitation and 617 nm emission (BioTek, Winooski, Vermont, United States).

### Data Analysis

Statistical analysis of all the results such as mean value, standard deviations, correlation (R2), significance and graphing were performed using the GraphPad Prism v.9.3.1 software. The results were statistically compared using the ordinary one-way Analysis of Variance (ANOVA) and two-way ANOVA, followed by multiple comparisons by Dunnett's post hoc test or Sidak's post hoc test. In all the graph plots, P values shown as *P<0.05, **P<0.01, ***P<0.001, and ****P<0.0001 were considered statistically significant, while n.s refers to non-significant.

### Example 1: Development of a Method for Quantification of Cell Viability Using Propidium Iodide Fluorescence Measurement in the Microplate Reader

A new protocol was developed where wild-type yeast cells or their mutated variants were added to 96-well plates with media enriched with nutrients and specified amounts of chemical agents of interest. Propidium iodide (PI) was utilized as the marker for cell death since this fluorescent dye only permeates through dead cells. Quantification of cell survival in the stationary phase after a specific number of days was done by reading the PI fluorescence data in a microplate reader, which then generated an electronically readable spreadsheet for data analysis. The total amount of cells was quantified by measuring the OD600 nm optical density from the same plate. A microplate reader is a low-cost device readily available in most laboratories, providing fast and efficient readout (within about 15 minutes) of absorbance and fluorescence.

It was first shown that carrying out the CLS study in microplate wells conveyed the same results as that in traditional methods using cuvettes, since the microplate reader is adequately sensitive to detect cell survival data via the fluorescence intensity measured. It was also necessary to determine if PI was an appropriate dye for staining the yeast cells. To test this, yeast cell cultures were grown in the glass flask and boiled for 15 minutes at 100° C., before both the live and dead cells were washed and incubated in 1×PBS under the presence and absence of PI (5 μg/mL) for 15 minutes. After incubation, the cells were washed and resuspended in PBS. Microscopic visualization of the cells was performed, and microplate readings of the fluorescence intensity were recorded (FIG. 3). This confirmed that PI was suitable as a fluorescent marker for cell death.

Next, PI-stained dead cells were resuspended in PBS to the final 48 OD600 nm measured in a cuvette using a spectrophotometer. The cells were then serially diluted in PBS (OD600 nm 48 to 0.05) before being transferred to a black 96-well plate (Costar 3603, Corning, Cambridge, Massachusetts, United States of America). Black microplates are four times more costly than clear plates and less easily available, but are more suitable for fluorescence measurements due to dampening of the autofluorescence originating from the samples and microplate surfaces. With a GelDoc imaging system, the degree of incorporation of PI staining in the dead yeast cells at various dilutions was visualized (FIG. 4A). Using the same plate, the PI fluorescence intensity was recorded using a microplate reader at 535 nm excitation and 617 nm emission wavelengths. This led to the identification of a high linear correlation between PI fluorescence intensity and cellular absorbance at OD600 nm (FIG. 4B). The linear correlation of the two properties indicated that this newly introduced PI fluorescence-based approach was effective for quantifying cell viability.

It was further necessary to identify the optimal range of cell density quantified by OD600 nm, so that cell density could be directly measured in 96-well plates instead of cuvettes. Using the black microplates with dead yeast cells as described above, the correlation of OD600 nm measurements taken from the cuvettes and from the microplate directly, was determined (FIG. 4C). A high linear correlation between the two series of measurements in the OD600 nm range 0.05 to 12 of the cuvette was found. However, this trend was no longer maintained for OD600 nm values greater than 12 in the cuvette measurements (FIG. 4D). Next, the relationship between PI fluorescence intensity and cell OD600 nm of the 96-well plate within the optimal OD600 nm range 0.05-12 was determined. An almost perfect linear correlation was observed (FIG. 4E). This emphasized the robustness of the inventive protocol for quantifying cell viability.

### Example 2: Effect of Microplate Types on Quantification of Cell Viability Using Propidium Iodide Fluorescence Measurement in the Microplate Reader

Although black microplates are recommended for fluorescence-based assays due to the ability to quench background fluorescence, their high cost and low availability called for cheaper and readily available alternatives. Comparisons were made between similar experiments carried out in both black and clear microplates (Costar 3596, Corning. Cambridge, Massachusetts, United States of America). PI-stained cells in clear microplates were visualized with a GelDoc imaging system to confirm sample staining (FIG. 5A). After confirmation by imaging, the PI fluorescence intensity was measured using the microplate reader and the relationship with cell OD600 nm values obtained for the cuvette measurements was determined. A high linear correlation between PI fluorescence intensity and cell OD600 nm intensity was found (FIG. 5B). A high correlation between cell OD600 nm values in the range 0.05 to 12 for the cuvette with the clear 96-well plate (FIG. 5C), and a distorted correlation for higher cell OD600 nm values greater than 12 of the cuvette was also found (FIG. 5D). The cell OD600 nm intensity of the clear 96-well plate was also found to be highly linearly correlated to its PI fluorescence intensity within the optimal range of 0.05 to 12 (FIG. 5E). Upon comparison of this set of results with those of the black 96-well plates, no significant differences were observed (FIGS. 6A, 6B, 6C and 6D). These results confirmed that the use of clear microplates was appropriate for this protocol.

### Example 3: Effect of Rapamycin on the Chronological Lifespan of the Yeast

Rapamycin is one of the well-known anti-aging agent that demonstrates the extension of lifespan of several model organisms, including yeast, nematodes, fruit flies and mice, by inhibiting the nutrient-sensing complex TORC1 (target of rapamycin complex 1). TORC1 is a conserved multi-subunit protein complex in eukaryotic cells that couples nutrients in the environment with cell growth and proliferation.

The effect of rapamycin on the CLS of a yeast strain was studied to examine the validity of the inventive protocol. Prototrophic yeast strain (CEN.PK113-7D) was utilized to circumvent the strong effects of amino acid auxotrophy on cell survival in stationary phase culture. Cells were aged with different concentrations of rapamycin in the SD medium and their respective growth at different time points (24 hours, 48 hours and 72 hours) were measured. It was found that cell growth reached saturation approximately 24 hours after incubating with 5 nM or less rapamycin (FIG. 7A). Administering 10 nM of rapamycin slowed cell growth, where saturation was achieved only after 48 hours. This trend has been previously observed for cells growing with rapamycin in a CLS experiment.

By utilizing PI fluorescence to determine cell survival at various chronological age time points, the CLS of aging cells grown in different concentrations of rapamycin was evaluated. The 72 hour growth time point was taken as day 1. Based on the survival graph plotted (FIG. 7B), it was deduced that different rapamycin concentrations extended the CLS of yeast. With higher concentrations of rapamycin (5 nM and 10 nM), the survival of aged cells on day 4 was approximately 75%, while those without rapamycin was approximately 50%. The difference was more significant on day 7, where cells incubated with rapamycin had a survival of approximately 70%, while those without rapamycin had survival of less than 20%. It was further observed that even with lower concentrations of rapamycin (0.62 to 2.5 nM), CLS extension of the aged cells occurred.

### Comparative Example 1: Evaluation of Cellular Lifespan Using Traditional Outgrowth Assay and Spot Assay

The inventive protocol was validated by directly comparing it with two traditional outgrowth assays that measures the CLS of yeast. The outgrowth assay was performed in liquid medium to assess the effect of rapamycin on CLS. The survival of chronologically aging cells was monitored at various age points by transferring a 3 μL cell culture into 200 μL of YPD medium in a fresh 96-well plate. Outgrowth correlated with the number of viable cells in the inoculum (FIG. 7C). A microplate reader was used to measure aged cell outgrowth via OD600 nm absorbance. The survival fraction was calculated from the outgrowth OD600 nm absorbance for each age-point relative to day 1 (considered 100% cell survival). The survival graph is shown in FIG. 7D.

Another method to assess the outgrowth of aged cells was to perform a spotting assay on the agar medium. After spotting 3 μL aging cell culture on the YPD agar plate and incubating for 48 hours, the GelDoc imaging system was utilized to visualize the outgrowth of the aged cells (FIG. 7E).

The inventive protocol was found to parallel the trends of CLS extension by rapamycin observed for both the YPD liquid and agar outgrowth assays.

These results validated the reproducibility of the protocol and the feasibility in using the inventive protocol for high-throughput screening. Furthermore, the Z-factor of the protocol was assessed to determine the quality of the inventive screening protocol. Generally, Z-factors of 0.5 to 1.0 suggest an excellent high-throughput assay. Hence, the Z-factor was evaluated for various concentrations of rapamycin with PI-stained aged cells (FIGS. 2A, 2B and 2C). Z-factors of 0.70 and 0.74 were obtained for 5 nM and 10 nM of rapamycin, respectively, putting this high-throughput screening method into the high-quality category. This validated that the inventive protocol was sufficiently robust for identifying potential anti-aging compounds.

### Example 4: Effect of Different Yeast Genetic Background

The inventive protocol was also tested on the BY4743 yeast strain, to determine whether it was still effective in a different yeast genetic background. The effect of rapamycin was tested on the CLS of BY4743 strain. Saccharomyces cerevisiae BY4743 strain is an auxotrophic for histidine, leucine and uracil. The BY4743 strain was grown in different concentrations of rapamycin in the SD medium supplemented with auxotrophic constituents (FIG. 8A). Survival of aged cells was quantified using PI fluorescence (FIG. 8B). It was found that rapamycin extended cell viability of the BY4743 cells similarly to that of CEN.PK113-7D. This implied the effectiveness of this protocol across various yeast strains.

### Example 5: Effect of Glucose on the Chronological Lifespan of the Yeast

Since calorie restriction has been known to slow aging and extend CLS of model organisms, including yeast and mammalian cells, the effect of calorie restriction on CLS of yeast was also investigated, to test the validity of the inventive protocol. This was done by restricting glucose content in the culture medium, where cells were cultured in three different SD medium conditions containing 0.25%, 0.5%, and 2% glucose. Cell growth was measured at various time points (24 hours, 48 hours, and 72 hours). It was observed that all cell cultures achieved growth saturation after 24 hours, but cell growth was notably lower in 0.25% and 0.5% glucose in comparison with that in 2% glucose (FIG. 9A). Survival of chronological aging for cells grown in different glucose concentrations was also evaluated using the PI fluorescence method. The survival graph implied that glucose restriction extended the CLS of yeast cells (FIG. 9B). Outgrowth assays concurrently confirmed this result (FIGS. 9C, 9D and 9E), validating the inventive protocol for determining the CLS of yeast. This inventive protocol also demonstrated the effect of calorie restriction on CLS extension in the BY4743 strain (FIGS. 8C and 8D). Hence, this approach for measuring the CLS of the yeast represents a novel, fast and low-cost method for screening the chemical agents and culturing conditions to identify the anti-aging interventions.

### Example 6: Screening and Identification of a Novel Anti-Aging Compound

As a proof of principle, hundreds of chemical agents were screened for the identification of anti-aging compounds (FIGS. 10A and 10B; Table 1) that may extend the CLS of yeasts (FIG. 1A) using the PI method. Of these, the compound 2,5-anhydro-D-mannitol (2,5-AM) was surprisingly shown to extend the CLS of yeasts.

TABLE 1
List of chemicals screened.

| | | | Concentration | | PI | | Yeast CLS |
|---|---|---|---|---|---|---|---|
| Number | Chemical name | Solvent | tested | | intensity | Outgrowth | increase |
| 1 | Aucubin | DMSO | 100 | μM | 58,414.0 | 0.1 | No |
| 2 | D-Glucuronic acid | DMSO | 100 | μM | 48,839.6 | 0.1 | No |
| 3 | β-Lapachone | DMSO | 100 | μM | 67,892.9 | 0.1 | No |
| 4 | Isocytosine | DMSO | 100 | μM | 68,813.6 | 0.1 | No |
| 5 | Rifamycin (sodium) | DMSO | 100 | μM | 39,446.0 | 0.6 | Yes |
| 6 | Cryptochlorogenic acid | DMSO | 100 | μM | 65,040.0 | 0.1 | No |
| 7 | Isochlorogenic acid A | DMSO | 100 | μM | 63,157.7 | 0.1 | No |
| 8 | Rhynchophylline | DMSO | 100 | μM | 55,562.5 | 0.1 | No |
| 9 | Mupirocin | DMSO | 100 | μM | 68,477.7 | 0.1 | No |
| 10 | 3,5-Dihydroxybenzoic acid | DMSO | 100 | μM | 59,369.7 | 0.1 | No |
| 11 | Ethylparaben | DMSO | 100 | μM | 52,097.7 | 0.1 | No |
| 12 | Nudifloramide | DMSO | 100 | μM | 65,023.3 | 0.1 | No |
| 13 | 4-Methoxycinnamic acid | DMSO | 100 | μM | 89,890.5 | 0.1 | No |
| 14 | Cefoselis (sulfate) | DMSO | 100 | μM | 70,425.6 | 0.1 | No |
| 15 | DL-Serine | DMSO | 100 | μM | 33,155.7 | 1.1 | Yes |
| 16 | Avibactam (sodium) | DMSO | 100 | μM | 73,562.3 | 0.1 | No |
| 17 | (S)-2-Hydroxy-3- | DMSO | 100 | μM | 72,969.5 | 0.1 | No |
| | phenylpropanoic acid | | | | | | |
| 18 | Vincristine (sulfate) | DMSO | 100 | μM | 62,812.1 | 0.1 | No |
| 19 | N-Acetyl-L-tyrosine | DMSO | 100 | μM | 57,322.5 | 0.1 | No |
| 20 | Rubitecan | DMSO | 100 | μM | 57,075.3 | 0.1 | No |
| 21 | Royal Jelly acid | DMSO | 100 | μM | 65,406.1 | 0.1 | No |
| 22 | β-Estradiol 17-acetate | DMSO | 100 | μM | 34,621.0 | 0.5 | Yes |
| 23 | 3,4-Dimethoxycinnamic | DMSO | 100 | μM | 70,914.2 | 0.1 | No |
| | acid | | | | | | |
| 24 | 2-Naphthol | DMSO | 100 | μM | 72,298.9 | 0.1 | No |
| 25 | N2,N2-Dimethylguanosine | DMSO | 100 | μM | 65,619.9 | 0.1 | No |
| 26 | Proxyphylline | DMSO | 100 | μM | 72,232.1 | 0.1 | No |
| 27 | Amarogentin | DMSO | 100 | μM | 70,083.5 | 0.1 | No |
| 28 | Lappaconitine | DMSO | 100 | μM | 76,000.0 | 0.1 | No |
| | (hydrobromide) | | | | | | |
| 29 | Rapamycin | DMSO | 100 | μM | 21,460.5 | 1.3 | Yes |
| 30 | 4-Ethylphenol | DMSO | 100 | μM | 58,041.7 | 0.1 | No |
| 31 | Purine | DMSO | 100 | μM | 53,185.1 | 0.1 | No |
| 32 | (S)-Indoximod | DMSO | 100 | μM | 78,648.2 | 0.1 | No |
| 33 | Actinonin | DMSO | 100 | μM | 75,254.2 | 0.1 | No |
| 34 | Cirsimaritin | DMSO | 100 | μM | 85,017.1 | 0.1 | No |
| 35 | 6-Biopterin | DMSO | 100 | μM | 75,974.5 | 0.1 | No |
| 36 | Hederagenin | DMSO | 100 | μM | 73,281.0 | 0.1 | No |
| 37 | Pentadecanoic acid | DMSO | 100 | μM | 75,173.9 | 0.1 | No |
| 38 | Staurosporine | DMSO | 100 | μM | 67,250.9 | 0.1 | No |
| 39 | Xanthoxylin | DMSO | 100 | μM | 70,352.1 | 0.1 | No |
| 40 | D-(+)-Cellobiose | DMSO | 100 | μM | 69,857.4 | 0.1 | No |
| 41 | Escin | DMSO | 100 | μM | 70,495.1 | 0.1 | No |
| 42 | 5α-Cholestan-3-one | DMSO | 100 | μM | 62,022.6 | 0.1 | No |
| 43 | 20(S)-Hydroxycholesterol | DMSO | 100 | μM | 75,450.9 | 0.1 | No |
| 44 | Anthraquinone-2-carboxylic | DMSO | 100 | μM | 96,099.2 | 0.1 | No |
| | acid | | | | | | |
| 45 | L-Kynurenine | DMSO | 100 | μM | 72,824.1 | 0.1 | No |
| 46 | Quinine (hydrochloride | DMSO | 100 | μM | 75,648.7 | 0.1 | No |
| | dihydrate) | | | | | | |
| 47 | DL-3-Phenyllactic acid | DMSO | 100 | μM | 65,266.9 | 0.1 | No |
| 48 | Retinoic acid | DMSO | 100 | μM | 85,242.6 | 0.1 | No |
| 49 | Schisantherin B | DMSO | 100 | μM | 68,765.7 | 0.1 | No |
| 50 | Narciclasine | DMSO | 100 | μM | 67,161.8 | 0.1 | No |
| 51 | Doripenem (monohydrate) | DMSO | 100 | μM | 69,608.7 | 0.1 | No |
| 52 | Diflorasone | DMSO | 100 | μM | 71,051.3 | 0.1 | No |
| 53 | Chlorogenic acid | DMSO | 100 | μM | 66,593.5 | 0.1 | No |
| 54 | Glucosamine | DMSO | 100 | μM | 65,905.5 | 0.1 | No |
| | (hydrochloride) | | | | | | |
| 55 | Gardenoside | DMSO | 100 | μM | 65,525.5 | 0.1 | No |
| 56 | Aloin B | DMSO | 100 | μM | 89,712.0 | 0.1 | No |
| 57 | 4-Methylumbelliferone | DMSO | 100 | μM | 90,234.6 | 0.1 | No |
| 58 | Lanolin | DMSO | 100 | μM | 84,056.6 | 0.1 | No |
| 59 | I-pyrrolidine-2-carboxylic | DMSO | 100 | μM | 80,551.7 | 0.1 | No |
| | acid | | | | | | |
| 60 | Glycodeoxycholic acid | DMSO | 100 | μM | 73,459.3 | 0.1 | No |
| | (monohydrate) | | | | | | |
| 61 | (−)-Epigallocatechin | DMSO | 100 | μM | 62,025.6 | 0.1 | No |
| 62 | 2′-Deoxyadenosine | DMSO | 100 | μM | 70,796.4 | 0.1 | No |
| 63 | Wogonin | DMSO | 100 | μM | 76,348.2 | 0.1 | No |
| 64 | Schisandrol B | DMSO | 100 | μM | 80,051.7 | 0.1 | No |
| 65 | 2,4-Dihydroxybenzoic acid | DMSO | 100 | μM | 76,326.3 | 0.1 | No |
| 66 | Hederacoside C | DMSO | 100 | μM | 73,515.9 | 0.1 | No |
| 67 | Noricaritin | DMSO | 100 | μM | 77,179.8 | 0.1 | No |
| 68 | Canrenone | DMSO | 100 | μM | 64,295.2 | 0.1 | No |
| 69 | Glycoursodeoxycholic acid | DMSO | 100 | μM | 64,205.2 | 0.1 | No |
| 70 | Glycodeoxycholic Acid | DMSO | 100 | μM | 68,981.6 | 0.1 | No |
| 71 | Cilastatin | DMSO | 100 | μM | 55,728.9 | 0.1 | No |
| 72 | Emetine (dihydrochloride | DMSO | 100 | μM | 75,341.5 | 0.1 | No |
| | hydrate) | | | | | | |
| 73 | Norgestrel | DMSO | 100 | μM | 75,338.8 | 0.1 | No |
| 74 | Heterophyllin B | DMSO | 100 | μM | 61,587.8 | 0.2 | No |
| 75 | Hispidin | DMSO | 100 | μM | 67,098.8 | 0.1 | No |
| 76 | Cinnamic acid | DMSO | 100 | μM | 60,256.3 | 0.1 | No |
| 77 | Catechin | DMSO | 100 | μM | 63,589.5 | 0.1 | No |
| 78 | [6]-Gingerol | DMSO | 100 | μM | 61,395.3 | 0.1 | No |
| 79 | Epiberberine (chloride) | DMSO | 100 | μM | 38,027.2 | 1.3 | Yes |
| 80 | Terconazole | DMSO | 100 | μM | 72,350.0 | 0.1 | No |
| 81 | Isoalantolactone | DMSO | 100 | μM | 94,566.8 | 0.1 | No |
| 82 | 2-Furoic acid | DMSO | 100 | μM | 72,748.2 | 0.1 | No |
| 83 | Vanillic acid | DMSO | 100 | μM | 64,546.4 | 0.1 | No |
| 84 | 5-Acetylsalicylic acid | DMSO | 100 | μM | 63,538.6 | 0.1 | No |
| 85 | Guggulsterone | DMSO | 100 | μM | 63,881.9 | 0.1 | No |
| 86 | Thymol | DMSO | 100 | μM | 61,503.8 | 0.1 | No |
| 87 | Homovanillic acid | DMSO | 100 | μM | 65,033.1 | 0.1 | No |
| 88 | Uridine | DMSO | 100 | μM | 84,894.9 | 0.1 | No |
| 89 | Gramicidin | DMSO | 100 | μM | 79,922.3 | 0.1 | No |
| 90 | Gossypol | DMSO | 100 | μM | 65,220.1 | 0.1 | No |
| 91 | Cyanidin-3-O-galactoside | DMSO | 100 | μM | 63,761.1 | 0.1 | No |
| | (chloride) | | | | | | |
| 92 | Kaempferol 3-O-β-D- | DMSO | 100 | μM | 62,874.6 | 0.1 | No |
| | glucuronide | | | | | | |
| 93 | L-Homoserine | DMSO | 100 | μM | 60,922.1 | 0.1 | No |
| 94 | D-Ribose(mixture of | DMSO | 100 | μM | 60,023.1 | 0.1 | No |
| | isomers) | | | | | | |
| 95 | Harmane | DMSO | 100 | μM | 71,690.9 | 0.1 | No |
| 96 | Geraniol | DMSO | 100 | μM | 76,340.1 | 0.1 | No |
| 97 | 3,4-Dimethoxyphenylacetic | DMSO | 100 | μM | 70,394.7 | 0.1 | No |
| | acid | | | | | | |
| 98 | 2-Phenylpropionic acid | DMSO | 100 | μM | 66,998.4 | 0.1 | No |
| 99 | Deltonin | DMSO | 100 | μM | 62,357.6 | 0.1 | No |
| 100 | 2-Oxovaleric acid | DMSO | 100 | μM | 61,246.4 | 0.1 | No |
| 101 | Estrone sulfate (potassium) | DMSO | 100 | μM | 54,751.8 | 0.1 | No |
| 102 | I-Citronellol | DMSO | 100 | μM | 75,290.6 | 0.1 | No |
| 103 | Euphorbia Factor L2 | DMSO | 100 | μM | 68,871.8 | 0.1 | No |
| 104 | β-Aminopropionitrile | DMSO | 100 | μM | 65,865.6 | 0.1 | No |
| 105 | Bronopol | DMSO | 100 | μM | 79,284.9 | 0.1 | No |
| 106 | Lincomycin (hydrochloride | DMSO | 100 | μM | 67,751.3 | 0.1 | No |
| | monohydrate) | | | | | | |
| 107 | Tridecanedioic acid | DMSO | 100 | μM | 80,407.7 | 0.1 | No |
| 108 | Calcifediol | DMSO | 100 | μM | 60,747.4 | 0.2 | No |
| 109 | Melanin | DMSO | 100 | μM | 39,360.1 | 1.0 | Yes |
| 110 | Methyl acetylacetate | DMSO | 100 | μM | 64,956.3 | 0.1 | No |
| 111 | L-Cysteine (hydrochloride | DMSO | 100 | μM | 69,216.4 | 0.1 | No |
| | hydrate) | | | | | | |
| 112 | Impulsin | DMSO | 100 | μM | 71,903.7 | 0.1 | No |
| 113 | Formamide | DMSO | 100 | μM | 64,135.2 | 0.1 | No |
| 114 | 3-Chloro-L-tyrosine | DMSO | 100 | μM | 67,122.0 | 0.1 | No |
| 115 | Tetramethylpyrazine | DMSO | 100 | μM | 62,003.6 | 0.1 | No |
| 116 | Esculin | DMSO | 100 | μM | 72,890.7 | 0.1 | No |
| 117 | DL-Panthenol | DMSO | 100 | μM | 71,468.6 | 0.1 | No |
| 118 | Baimaside | DMSO | 100 | μM | 61,895.7 | 0.2 | No |
| 119 | Benzyl acetate | DMSO | 100 | μM | 74,321.9 | 0.1 | No |
| 120 | Gentiopicroside | DMSO | 100 | μM | 59,804.7 | 0.1 | No |
| 121 | Guaiazulene | DMSO | 100 | μM | 58,776.1 | 0.1 | No |
| 122 | Engeletin | DMSO | 100 | μM | 37,144.4 | 0.9 | Yes |
| 123 | Acivicin | DMSO | 100 | μM | 41,066.2 | 1.0 | Yes |
| 124 | Tenofovir (hydrate) | Water | 100 | μM | 76,065.0 | 0.1 | No |
| 125 | Metyrosine | Water | 100 | μM | 76,947.2 | 0.1 | No |
| 126 | Ripasudil | Water | 100 | μM | 77,962.0 | 0.1 | No |
| 127 | Diphenmanil (methylsulfate) | Water | 100 | μM | 78,623.4 | 0.1 | No |
| 128 | Ambroxol (hydrochloride) | Water | 100 | μM | 63,509.0 | 0.1 | No |
| 129 | VAL-083 | Water | 100 | μM | 63,325.5 | 0.1 | No |
| 130 | Aliskiren (hemiumarate) | Water | 100 | μM | 73,607.3 | 0.1 | No |
| 131 | Selenomethionine | Water | 100 | μM | 29,837.8 | 1.2 | Yes |
| 132 | Cisatracurium (besylate) | Water | 100 | μM | 75,862.3 | 0.1 | No |
| 133 | DL-Methionine | Water | 100 | μM | 66,281.7 | 0.1 | No |
| | methylsulfonium (chloride) | | | | | | |
| 134 | Biapenem | Water | 100 | μM | 71,409.3 | 0.1 | No |
| 135 | Dipotassium glycyrrhizinate | Water | 100 | μM | 68,519.8 | 0.1 | No |
| 136 | L-Ornithine (hydrochloride) | Water | 100 | μM | 64,179.1 | 0.1 | No |
| 137 | Fosphenytoin (disodium) | Water | 100 | μM | 61,860.0 | 0.1 | No |
| 138 | I-Baclofen | Water | 100 | μM | 61,049.3 | 0.1 | No |
| 139 | Trapidil | Water | 100 | μM | 56,443.9 | 0.1 | No |
| 140 | Xanthinol Nicotinate | Water | 100 | μM | 49,233.6 | 0.1 | No |
| 141 | Cerivastatin (sodium) | Water | 100 | μM | 64,250.5 | 0.1 | No |
| 142 | Aprotinin | Water | 100 | μM | 70,144.6 | 0.1 | No |
| 143 | Flavin adenine dinucleotide | Water | 100 | μM | 59,508.7 | 0.2 | No |
| | (disodium salt) | | | | | | |
| 144 | Biperiden (Hydrochloride) | Water | 100 | μM | 67,178.1 | 0.2 | No |
| 145 | Streptomycin (sulfate) | Water | 100 | μM | 60,638.5 | 0.1 | No |
| 146 | Cefadroxil | Water | 100 | μM | 70,326.1 | 0.1 | No |
| 147 | Diclofenac (Sodium) | Water | 100 | μM | 79,704.3 | 0.2 | No |
| 148 | Estramustine (phosphate | Water | 100 | μM | 77,514.0 | 0.2 | No |
| | sodium) | | | | | | |
| 149 | Amifostine | Water | 100 | μM | 69,013.2 | 0.2 | No |
| 150 | Quetiapine sulfoxide | Water | 100 | μM | 68,761.6 | 0.1 | No |
| | (dihydrochloride) | | | | | | |
| 151 | Minocycline (hydrochloride) | Water | 100 | μM | 71,078.2 | 0.1 | No |
| 152 | L-SelenoMethionine | Water | 100 | μM | 31,240.0 | 1.2 | Yes |
| 153 | Cangrelor (tetrasodium) | Water | 100 | μM | 83,173.6 | 0.1 | No |
| 154 | Chondroitin (sulfate) | Water | 30 | μg/mL | 75,572.6 | 0.1 | No |
| 155 | Hyaluronidase | Water | 30 | μg/mL | 78,406.7 | 0.1 | No |
| 156 | Heparin (Lithium salt) | Water | 30 | μg/mL | 77,829.1 | 0.1 | No |
| 157 | Heparin (sodium salt) | Water | 30 | μg/mL | 64,969.0 | 0.1 | No |
| | (MW 15 kDa) | | | | | | |
| 158 | Gentamicin (sulfate) | Water | 30 | μg/mL | 73,296.2 | 0.1 | No |
| 159 | Polidocanol | Water | 30 | μg/mL | 74,745.5 | 0.1 | No |
| 160 | 2,5-anhydromannitol | Water | 300 | μg/mL | 36,240.0 | 1.3 | Yes |
| 161 | DMSO control 1 | DMSO | | | 69,929.1 | 0.2 | |
| 162 | DMSO control 2 | DMSO | | | 70,278.0 | 0.1 | |
| 163 | DMSO control 3 | DMSO | | | 70,494.8 | 0.1 | |
| 164 | DMSO control 4 | DMSO | | | 67,035.1 | 0.1 | |
| 165 | DMSO control 5 | DMSO | | | 78,011.0 | 0.1 | |
| 166 | DMSO control 6 | DMSO | | | 68,377.5 | 0.1 | |
| 167 | DMSO control 7 | DMSO | | | 78,899.7 | 0.1 | |
| 168 | DMSO control 8 | DMSO | | | 73,013.1 | 0.1 | |
| 169 | Water control 1 | Water | | | 55,358.7 | 0.1 | |
| 170 | Water control 2 | Water | | | 76,405.5 | 0.1 | |
| 171 | Water control 3 | Water | | | 63,579.4 | 0.1 | |
| 172 | Water control 4 | Water | | | 62,094.3 | 0.1 | |
| 173 | Water control 5 | Water | | | 72,575.8 | 0.1 | |
| 174 | Water control 6 | Water | | | 83,192.6 | 0.1 | |
| 175 | Water control 7 | Water | | | 67,575.6 | 0.2 | |
| 176 | Water control 8 | Water | | | 69,669.4 | 0.1 | |

### Example 7: Cellular Lifespan Determination of Human Cells Using Propidium Iodide

The validity of the inventive protocol was also tested on human cells. Similar to previous examples, the fluorescence readings of the human cell line samples were measured by a microplate reader at 535 nm excitation and 617 nm emission on day 1, day 4, day 6, and day 8. Cell survival was determined by normalization of fluorescence intensity at different rapamycin concentrations with untreated control (DMSO) for each result on the respective days. Based on the results obtained, it was observed that rapamycin had increased the chronological lifespan of both HEK293 and A549 cells (FIGS. 11A and 11B, respectively). Hence, this implied that the inventive protocol was robust enough to identify potential anti-aging compounds in human cell lines.

In a further investigation using the PI assay, the anti-aging effect of 2,5-AM in the human lung primary fibroblast cells, IMR90, was also tested. The primary fibroblast cells are widely used in aging research as they have a finite lifespan, and they better mimic the organism's aging process compared to immortal cell lines. 2,5-AM treatment significantly increased the cellular lifespan of IMR90 cells, resulting in a lower percentage of cell death. Both the 2.5 mM and the 5 mM treatment groups showed 32.33% and 48.08% more live cells compared to the untreated control, respectively (FIG. 12A).

It was also found that 2,5-AM increased cellular lifespan in the HEK293 cells, compared to the untreated group (FIG. 12B). The 2.5 mM and 5 mM treatment experiments showed 34.39% and 58.7% more live cells, respectively (FIGS. 12A and 12B). Likewise, rapamycin application showed a similar trend in the HEK293 cell line (FIG. 13) The cellular viability using 2 μg/mL PI or 5 μg/mL of PI in HEK293 and IMR90 cells were also investigated. It was found that a similar pattern of anti-aging activities could be observed, despite variations in the method (FIG. 14).

The inventive protocol did not require any outgrowth procedures, including aspiration, washing, trypsinization or transferring of cells to a new plate. As such, the inventive protocol was found to be more economical and reduced variations introduced in the outgrowth methods such as those described in the Comparative Examples, as it required one plate per experiment only. The assay was easy to perform as no additional steps were required after the assay plate had been cultured, and readings could be taken at multiple age time points with the same samples on a microplate reader. The PI assay measured the amount of cell death. Hence, it was independent of the ability of the cells to proliferate. Therefore, it was also found to be suitable for compounds or procedural interventions that suspend cell proliferation.

In addition, the inventive protocol can be conducted with a large variety of human cell lines, including cells from various human tissues as well as cancer and senescence-induced cell lines. It may be useful to assess the anti-cancer or senolytic potential of large sets of compounds and interventions.

A schematic comparison of the PB outgrowth assay in Comparative Example 3 and the inventive protocol is shown in FIG. 15.

These advantages made the inventive protocol highly compatible with high-throughput compound screening requirements. The protocol is not just restricted to the CLS measurement alone. As the inventive protocol is not limited to the type of compounds used, it may be used for senolytic compound screening with senescent-induced cells or as anti-cancer compound screening with cancer cell lines under nutrient-limiting conditions. Thus, there is more potential in this cell viability assay with the usage of different cell lines. The HTS method utilizing the inventive protocol may be potentially valuable in many additional ways for drug discovery and biopharmaceutical research.

### Comparative Example 2: Evaluation of Human Cellular Lifespan Using the Crystal Violet Outgrowth Assay

In this approach, an outgrowth assay was used to determine the number of live cells in the 6-well experiment plate using crystal violet staining. Crystal violet is a triarylmethane dye that binds to ribose molecules such as DNA. The crystal violet-based outgrowth method relies on the detachment of adherent cells from the culture plate during cell death. Hence, dead cells would be washed away with the D10 medium and only live cells would remain attached to the culture plate. These live cells were then stained with crystal violet dye and assessed qualitatively.

The cell viability of 2,5-AM treated cells was examined at different concentrations using the crystal violet-based CLS assay. It was observed that after seven days of outgrowth. 2,5-AM was able to increase cellular lifespan in HEK293 cell line (FIG. 16A). Similar results could be observed on the Day 8 culture (FIG. 16C) but not on the Day 6 culture (FIG. 16B). This may be due to the variation in washing steps during the crystal violet staining.

However, this assay required a long time for cellular outgrowth. It also showed that there was a risk of cell detachment during multiple washing steps in the staining. It was not suitable for high-throughput screening (HTS) analysis of multiple compounds, concentrations and/or treatments. It was also evident that the qualitative nature of the obtained data could obscure analysis, resulting in ambiguous or misleading outcomes at a low number of surviving cells.

### Comparative Example 3: Evaluation of Cellular Lifespan Using the PrestoBlue™ Outgrowth Assay

In order to overcome some of the limitations mentioned in the crystal violet assay, a method that requires less time, has less experimental error and, most importantly, generates quantitative data for CLS analysis of human cells was explored. The anti-aging compound 2,5-AM, was used to validate the efficiency of this approach based on the outgrowth PrestoBlue™ assay. PrestoBlue (PB) is a resazurin-based reagent used to detect cell viability and traces of cytotoxicity in the medium. It produces results that can be detected both colorimetrically and fluorometrically. It is permeable and non-toxic to the cells, hence, enabling the measurement of cell viability over a period of time without cell lysis. The resazurin component is reduced to form resorufin by live cells, resulting in a visible change from blue colour to red colour in the reagent, at the same time developing fluorescence, which can be easily detected by a microplate reader. The PB reagent is highly sensitive; it detects even a small number of cells (a couple of hundreds). It allows conveniently fast cell detection and data collection. The PB assay was easy to perform as the reagent is a commercially available as ready-to-use solution. The method is quantitative, can be used in high-throughput analysis and has a fast output. It should also be noted that the PB can be replaced with other fluorescent viability dyes including propidium iodide, and various resazurins such as MTT, XTT, and alamarBlue, to obtain the same results.

In the outgrowth assay using human cell lines, 2,5-AM treatment significantly increased the cellular lifespan, which resulted in more live cells that were able to proliferate in the assay plate. For the HEK293 cell line, the 2.5 mM treatment group had 76.31% more viable cells compared to the untreated group (FIG. 17A). Moreover, the 5 mM treatment group had even 123.7% more surviving cells relative to the untreated group (FIG. 17A).

The anti-aging effect of 2,5-AM in the human lung primary fibroblast cells, IMR90 was tested. An anti-aging effect similar to that for HEK293 was also observed for the IMR90 cells. The 2.5 mM treatment group had 59.05% more surviving cells and the 5 mM treatment group had 59.89% more viable cells compared to the untreated group (FIG. 17B).

The cellular outgrowth for 5% (10 μL) cells transferred to the assay plates for the HEK293 and IMR90 cell were also examined. A pattern of anti-aging activities similar to the results when 10% of cells was transferred (FIG. 18) was also found. Thus, these results suggested that outgrowth time can be shorten by increasing the amount of transferred cells without affecting the results.

The PB-based outgrowth method was further tested with rapamycin, an established anti-aging compound in multiple models, including the HT-p21-9 cell assay where it has been shown to increase cellular lifespan. It was found that rapamycin treatment significantly increased the lifespan of HEK293 cells (FIG. 19). This further confirmed the robustness of the method to identify and to evaluate potential anti-aging compounds.

This assay allowed determination of cellular lifespan in a short time with significant throughput capacity. The detection of viable cells using the PB reagent provided quantitative data, which can be computationally further evaluated. Notably, other resazurin-based reagents such as the MTT, XTT and alamarBlue reagent can replace PB as well as any efficient colorant that stains surviving cells.

However, the outgrowth assay required aspiration of medium, washing and trypsinization, similarly to the outgrowth crystal violet assay. These steps introduced multiple sources of errors to the experiment such as cell detachment during aspiration and washing, potential problems during trypsinization or the transfer of live cells. To note, the outgrowth assay depended on the ability of the cells to proliferate in the fresh medium after days of compound treatment. Hence, if the treatment caused a decline in cell proliferation, cells would not proliferate even if they were alive, giving a false negative result. For example, calorie restriction has been shown to increase cellular lifespan but reduce proliferation. Thus, this outgrowth assay was not suitable for the detection of cellular lifespan in this scenario. Moreover, inefficiency in detecting surviving cells with low metabolic activity was also an issue with the PB assay and other related assays that measured cytotoxicity via a NADH-dependent cellular oxidoreductase enzymes.

### INDUSTRIAL APPLICABILITY

The method as defined above may be useful in high-throughput screening of a large number of potential chemical agents or culturing conditions which affect CLS in various types of eukaryotic cell models or human cell lines. The method may be useful in the quantification of CLS in said cell models. The method may be useful in high-throughput screening for the identification of geroprotective interventions or potential drugs within a short period of time at a considerably low cost. The method may be useful in the high-throughput screening of gene-drug interactions in an array of genetically modified strains. The method may be useful in the screening of gene-regulated age-dependent factors that affect CLS of cells. The method may be useful in identification of anti-aging compounds, toxic chemicals that accelerate aging and death, anti-fungal compounds, senolytics and anti-cancer agents.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-23. (canceled)

24. A method for high-throughput screening to determine cellular lifespan, the method comprising the steps of:
a) treating, in a well of a first multi-well plate, a sample containing cells with a condition that modulates lifespan of cells;
b) transferring a portion of the cells in the well of the first multi-well plate into a well of a second multi-well plate;
c) mixing, in a well of the second multi-well plate, the portion of cells with a fluorescent viability dye to stain the cells;
d) measuring fluorescence intensity of the stained cells in a plate reader to obtain a fluorescence intensity value;
e) measuring optical density of the cells in the plate reader to obtain an optical density value; and
f) normalizing the fluorescence intensity value with the optical density value, wherein the normalized fluorescence intensity value directly correlates with the viability of the cells.

25. The method according to claim 24, wherein the condition that modulates the cellular lifespan of cells is exposure to a lifespan modulating agent, or exposure to environmental conditions that modulate the lifespan of cells.

26. The method according to claim 25, wherein the cellular lifespan modulating agent is selected from the group consisting of an anti-aging agent, anti-microbial agent, anti-fungal agent, anti-bacterial agent, anti-cancer agent, pharmaceutical drug, genotoxic agents, toxic chemical agent, and any mixture thereof.

27. The method according to claim 25, wherein the conditions that modulate the lifespan of cells is level of nutrients including carbohydrates, proteins, and lipids, carbon, nitrogen, amino acids, phosphate, nucleosides, nucleotides, nutrient starvation, osmotic condition, oxidative stress, temperature, pH, oxygen concentration, and any mixture thereof.

28. The method according to claim 24, wherein the treating step (a) is performed in a first multi-well plate, wherein the first multi-well plate of step (a) is different to the second multi-well plate of step (c).

29. The method according to claim 24, wherein the treating step (a) comprises a step (a1) of treating the cells with a lifespan modulating agent at various concentrations or a step (a2) of exposing the cells to a condition that modulates lifespan for a various duration.

30. The method according to claim 24, wherein the transferring step (b) comprises the step (b1) of shaking the first multi-well plate to form a cell suspension.

31. The method according to claim 24, wherein:
the multi-well plate is a 6-well plate, 12-well plate, 24-well plate, 48-well plate, 96-well plate, 384-well plate or a 1536-well plate, or is a clear plate or an opaque-walled plate;
the sample containing the cells comprises cell culture media or phosphate buffered saline; and/or
the cells are selected from the group consisting of fungal cells, bacterial cells and animal cells.

32. The method according to claim 31, wherein the multi-well plate is a 96-well plate, and the sample containing cells comprises 30 L to 200 μL of cell culture media or phosphate buffered saline.

33. The method according to claim 24 wherein the mixing step (c) comprises a step (c1) of shaking the second multi-well plate.

34. The method according to claim 24, wherein the fluorescent viability dye is selected from the group consisting of propidium iodide, PrestoBlue™, 3-(4,5-Dimethylthiazol-2-yl)-2,5-Diphenyltetrazolium Bromide (MTT), 2,3-Bis-(2-Methoxy-4-Nitro-5-Sulfophenyl)-2H-Tetrazolium-5-Carboxanilide (XTT), alamarBlue reagent, 7-hydroxy-10-oxidophenoxazin-10-ium-3-one (resazurin), SYTO 9, 2-chloro-4-(2,3-dihydro-3-methyl-(benzo-1,3-thiazol-2-yl)-methylidene)-1-phenylquinolinium iodide (Fun 1), disodium 2,2′-ethene-1,2-diylbis [5-({4-anilino-6-[bis(2-hydroxyethyl)amino]-1,3,5-triazin-2-yl}amino)benzenesulfonate] (Calcofluor White), 3′,6′-Bis(acetyloxy)spiro[isobenzofuran-1 (3H), 9′-[9H]xanthen]-3-one (fluorescein diacetate), N,N,N′,N′-Tetramethyl-3,6-acridinediamine (acridine orange), 2′-(4-Ethoxyphenyl)-5-(4-methyl-1-piperazinyl)-2,5′-bi-1H-benzimidazole trihydrochloride (Hoechst 33342) and any combination thereof.

35. The method according to claim 24, wherein in the mixing step (c), the fluorescent viability dye is propidium iodide and the propidium iodide is present at a concentration in the range of 3 μg/mL to 7 μg/mL in phosphate buffered saline.

36. The method according to claim 24, further comprising a step (x1) of washing the cells after the transfer step (b) and before the mixing step (c), or a step (x2) of washing the cells after the mixing step (c) and before the measuring step (d).

37. The method according to claim 36, wherein the washing steps (x1) and (x2) independently comprise the steps of:
y1) centrifuging the multi-well plate to separate the cells from the cell culture media, phosphate buffered saline, residual fluorescent viability dye and any mixture thereof;
y2) removing the cell culture media, phosphate buffered saline, residual fluorescent viability dye and any mixture thereof from the cells; and
y3) resuspending the cells in phosphate buffered saline.

38. The method according to claim 36, wherein the washing steps (x1) and (x2) are independently to be repeated multiple times.

39. The method according to claim 24, wherein the method steps (c) to (f) are performed within a duration of less than 45 minutes.

40. The method according to claim 24, further comprising a step (g) of comparing the normalized fluorescence intensity value of cells treated with the lifespan modulating condition with the fluorescence intensity value of positive control cells and negative control cells.

41. The method according to claim 40, wherein the comparing step (g) further comprises a step (g1) of calculating % cell survival.

42. The method according to claim 24, comprising the steps of:
a) treating, in a well of a first multi-well plate, a sample containing cells with an anti-aging agent;
b) transferring a portion of the cells in the well of the first multi-well plate into a well of a second multi-well plate;
x1) optionally washing the cells;
c) mixing, in the well of the second multi-well plate, the portion of the cells with a fluorescent viability dye to stain the cells;
x2) optionally washing the stained cells;
d) measuring fluorescence intensity of the stained cells in a plate reader to obtain a fluorescence intensity value;
e) measuring optical density of the stained cells in the plate reader to obtain an optical density value;
f) normalizing the fluorescence intensity value with the optical density value, wherein the normalized fluorescence intensity value directly correlates with the viability of the cells;
g) comparing the normalized fluorescence intensity value of cells treated with the anti-aging agent with the fluorescence intensity value of positive control cells and negative control cells;
g1) calculating the % cell survival.

43. A method for high-throughput screening to determine cellular lifespan, the method comprising the steps of:
a) plating, in a well of the multi-well plate, a sample containing cells and a fluorescent viability dye to stain the cells,
b) treating, in the well of the multi-well plate, the sample containing cells with a condition that modulates lifespan of cells; and
c) measuring fluorescence intensity of the stained cells in a plate reader to obtain a fluorescence intensity value.
